(19) United States
(12) Patent Application Publication
(10) Pub. No.: US 2026/0250689 A1
(43) Pub. Date: Aug. 27, 2026

(54) MEMBRANE ENCAPSULATED CELL-FREE SYSTEMS WITH BIOSENSING CAPABILITIES

(71) Applicant: Northwestern University, Evanston, IL (US)

(72) Inventors: Margrethe Boyd, Evanston, IL (US); Walter Math Thavarajah, Evanston, IL (US); Julius Beau Lucks, Evanston, IL (US); Neha Prashant Kamat, Evanston, IL (US)

(21) Appl. No.: 19/161,065

(22) PCT Filed: Mar. 1, 2024
(86) PCT No.: PCT/US2024/018071

§ 371 Date: Aug. 29, 2025

Related U.S. Application Data
(60) Provisional application No. 63/487,864, filed on Mar. 1, 2023.

(51) Int. Cl. C12N 15/115; G01N 33/18

(52) U.S. Cl. CPC C12N 15/115; G01N 33/18; C12N 2310/16; C12N 2310/3513

(57) ABSTRACT

Provided herein are membrane encapsulated cell-free systems comprising transcriptionally regulated biosensors for detecting analytes, and methods of their use. In embodiments, the biosensor is a riboswitch for detection of fluoride in a sample.

### CROSS-REFERENCE TO RELATED APPLICATIONS

This patent application claims the benefit of, and priority to, U.S. Provisional Patent Application No. 63/487,864, filed Mar. 1, 2023. The content of which is incorporated herein by reference in its entirety.

### STATEMENT REGARDING FEDERALLY SPONSORED RESEARCH

This invention was made with government support under DMR-2145050, CBET-1844219 and CBET-1844336 awarded by the National Science Foundation. The government has certain rights in the invention.

### BACKGROUND OF THE INVENTION

Cell-free systems have emerged as a powerful technology to detect a wide variety of molecular signals, including chemical contaminants relevant to the environment and human health and markers of disease and infection. By reconstituting purified cellular machinery in vitro, these systems enable use of natural cellular biochemical sensing mechanisms in a low-cost, distributable, and easily tunable platform. Despite these key advantages, removal from the cell also eliminates certain features of the cell's native membrane barrier-such as reaction containment, protection from reaction inhibitors, and selective gating-all of which can add important functionality to cell-free biosensors.

Efforts to deploy sensors highlight the limitations caused by the absence of cellular membranes. For example, without a barrier between the sensor and the sample, detecting targets in complex matrices like polluted water or biological samples requires additional modifications to the reaction or preparation protocols. This is because cell-free reactions are easily inhibited by changes in salt concentrations, the presence of detergent-like molecules, as well as bioactive enzymes like DNA-/RNAses that either degrade or compete with components of the reaction. Cell-free sensors are also sensitive to dilution, and therefore require a controlled reaction environment. One strategy to mitigate these limitations is to recapitulate some of the lost features of the cell membrane by encapsulating cell-free sensors inside of synthetic membranes. These structures, often referred to as artificial cells, leverage powerful cellular protein expression mechanisms inside a cell-like compartment in order to carry out dedicated sensing behaviors. In these systems, encapsulation in a semipermeable compartment allows users to tune the reaction environment on a molecular scale, enabling control over molecular access to the sensor, all while maintaining many of the advantageous features of cell-free systems.

There are two major considerations in designing artificial cell-based sensors: determining the impacts of a confined reaction environment on sensor function and choosing an appropriate target molecule and application. In terms of reaction confinement, the small scale of the encapsulated environment can impact reactant loading, reaction time, and limit of detection. These effects have been shown to impact the basic processes of gene expression, which in turn affects cell-free biosensors that regulate reporter gene expression at the level of transcription or translation. Of the wide range of genetic regulatory networks used for biosensing, RNA-based biosensors that regulate transcription (mRNA synthesis) require the fewest components and operate on a faster timescale, which may reduce the impacts of confinement on sensor function relative to biosensors that regulate translation (protein synthesis). Riboswitches-noncoding RNA elements upstream of protein coding genes that change their structure in response to specific ligands to regulate gene expression-could offer an opportunity to address these constraints due to reaction confinement.

Previous proof-of-concept studies have focused on encapsulation of two synthetic, translationally regulated riboswitches that respond to membrane-permeable signals: theophylline and histamine. Both riboswitches have been successfully encapsulated in bilayer vesicles, generating either a fluorescent protein readout or a protein-mediated response upon analyte entry into the vesicle interior. Encapsulation of transcriptionally regulated riboswitches has yet to be demonstrated. Transcriptional riboswitches require dynamic conformational changes during transcription to enact their mechanism—a process which could be impacted by general features of confinement or electrostatic interactions with the lipid bilayers. Despite these potential challenges, the mechanisms underlying transcriptionally regulated riboswitches are being further uncovered. Importantly, these sensors have demonstrated the feasibility of detecting environmentally important analytes in cell-free systems and can function with RNA-level outputs—a key feature which may mitigate resource constraints-motivating further efforts for their encapsulation and deployment.

A second major consideration in encapsulated sensor development is the selection of an appropriate target and application. Of the many potential uses of encapsulated biosensors, water quality monitoring is one of the most compelling from a global perspective. One in three people globally lack access to safe drinking water, and the ability to identify contaminated water sources is essential for their quarantine or remediation. Fluoride is among the most concerning of these contaminants; chronic exposure to fluoride binds it to the calcium in teeth and bones, weakening them and causing lifelong health consequences. From both environmental and anthropogenic sources, fluoride exposure is especially problematic in parts of China, Africa, South America, and India, with high fluoride concentrations also found in groundwater across the United States. This diversity of sample sources comes with a corresponding increase in potential reaction inhibitors, presenting the need for a robust sensor that retains function in complex matrices. Encapsulated fluoride biosensing reactions would address this need, delivering far-reaching global health benefits and establishing a framework to address future water quality challenges.

### SUMMARY OF THE INVENTION

In an aspect, provided herein composition comprising: a cell-free system; and a construct encoding a riboswitch coupled to a reporter protein; wherein the riboswitch is responsive to an analyte; and wherein the composition is encapsulated within a membrane. In embodiments, the membrane is a bilayer membrane. In embodiments, the membrane comprises 1-palmitoyl-2-oleoyl-glycero-3-phosphocholine (POPC). In embodiments, the membrane further comprises at least one of cholesterol, oleic acid, and poly(ethylene oxide)-b-poly(butadiene) (PEO-b-PBD) diblock copolymer. In embodiments, the membrane comprises a 2:1 cholesterol:POPC lipid composition.

In embodiments, the analyte is fluoride. In embodiments, the riboswitch is a Bacillus cereus riboswitch.

In embodiments, the reporter protein is a fluorescent reporter or a colorimetric reporter. In embodiments, the reporter protein is a fluorescent reporter, and the composition further comprises a volume marker. In embodiments, the reporter protein is green fluorescent protein (GFP). In embodiments, the reporter protein is GFP, and the volume marker comprises OA647. In embodiments, the reporter protein is catechol (2,3)-dioxygenase (C23DO), and the composition further comprises catechol. In embodiments, the composition comprises about 1 mM catechol.

In another aspect, disclosed herein is a method of detecting an analyte in a sample, the method comprising: contacting the sample with a composition comprising a cell-free system and a construct encoding a riboswitch coupled to a reporter protein, wherein the composition is encapsulated within a membrane; and detecting a signal emitted by the reporter protein; wherein the riboswitch is responsive to the analyte; and wherein if the analyte is present in the sample, the signal is emitted. In embodiments, the membrane is a bilayer membrane. In embodiments, the membrane comprises 1-palmitoyl-2-oleoyl-glycero-3-phosphocholine (POPC). In embodiments, the membrane further comprises at least one of cholesterol, oleic acid, and poly(ethylene oxide)-b-poly(butadiene) (PEO-b-PBD) diblock copolymer. In embodiments, the membrane comprises a 2:1 cholesterol:POPC lipid composition.

In embodiments, the analyte is fluoride, and the riboswitch is a fluoride-responsive riboswitch. In embodiments, the fluoride-responsive riboswitch is a Bacillus cereus riboswitch. In embodiments, the reporter protein is a fluorescent reporter or a colorimetric reporter. In embodiments, the reporter protein is a fluorescent reporter, and the composition further comprises a volume marker. In embodiments, the reporter protein is green fluorescent protein (GFP). In embodiments, the reporter protein is GFP; and the volume marker is OA647. In embodiments, the reporter protein is catechol (2,3)-dioxygenase (C23DO), and wherein the composition further comprises catechol. In embodiments, the composition comprises about 1 mM catechol.

In embodiments, the method further comprises incubating the sample in the system for at least 90 minutes before detecting the signal. In embodiments, the method further comprises incubating the sample in the system for at least three hours before detecting the signal. In embodiments, the method further comprises incubating the sample in the system for between about three and about five hours before detecting the signal. In embodiments, the method further comprises adding an RNAse to the sample before contacting the sample with the composition. In embodiments, the sample is a water sample.

In another aspect, provided herein is a kit comprising any of the compositions described herein, wherein the composition is in a container that a sample can be added to. In embodiments, the container is a clear container, and the reporter protein is a colorimetric reporter that emits a signal visible to the eye.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Artificial cell sensors. Encapsulation of cell-free systems in artificial cells creates a semipermeable barrier between sensor components and the environment, which modulates their molecular interactions. Reactants are contained within the vesicle interior, while proteins and other large molecules in the external sample are excluded from vesicle entry (top). Small, membrane-permeable molecules can diffuse into the vesicle interior, initiating a riboswitch-mediated response that is specific to an analyte of interest (bottom right). The riboswitch folds into a terminating conformation in the absence of sufficient concentrations of target analyte (bottom left).

FIGS. 2A-2C. A fluoride riboswitch can function within bilayer vesicles. FIG. 2A. Representative trace of riboswitch-regulated GFP expression in bulk conditions in response to increasing fluoride concentrations. In the presence of NaF the riboswitch folds into an “ON” state, which allows expression of a GFP reporter molecule. FIG. 2B. Double emulsion assembly allows the encapsulation of functional cell-free reactions. Assembled reactions are vortexed into a lipid/oil mixture, then centrifuged into an aqueous solution (left). The resulting vesicles contain cell-free reactions which can respond to co-encapsulated fluoride by expressing GFP (right). FIG. 2C. GFP/OA647 fluorescence, which indicates GFP concentration relative to the OA647 volume marker inside each liposome. GFP/OA647 fluorescence increases inside of vesicles when 3 mM NaF is co-encapsulated compared to no DNA (Extract) or no fluoride (0 mM NaF) controls. Micrographs show variations in GFP fluorescence between vesicles from the same population, which results in a distribution of fluorescence values. Scale=50 μm. Black lines indicate mean fluorescence and standard deviation. **** p≤0.0001, nonsignificant (ns) p>0.05; p-values generated using a One-Way ANOVA and Tukey's Multiple Comparisons Test.

FIGS. 3A-3E. Encapsulated sensors detect external fluoride. FIG. 3A. Schematic of conditions. Vesicles were prepared encapsulating extract only (left), or fully assembled reactions without NaF. Upon addition of increasing fluoride in the external solution, expression of GFP inside vesicles increases (right). FIG. 3B. GFP/OA647 fluorescence as a result of riboswitch activity in 2:1 cholesterol:POPC vesicles in response to increasing NaF added externally. Black lines indicate mean fluorescence ratio and standard deviation. FIG. 3C. Histogram of vesicle populations shown in FIG. 3B. Data plotted with lowless curve fitting. FIG. 3D. GFP fluorescence in micrographs of vesicles with increasing external concentrations of NaF. Scale=50 μm. FIG. 3E. GFP/OA647 fluorescence in response to increasing fluoride shown from left to right: 2:1 cholesterol:POPC membranes; pure POPC lipid membranes; POPC+10% oleic acid membranes; POPC+10% 1.8k PEO-b-PBD membranes. Composition and morphology of each membrane composition indicated by schematics and micrographs, respectively. **** p≤0.0001, ** p≤0.01, * p≤0.05, nonsignificant (ns) p>0.05; p-values generated using One-Way ANOVA and Tukey's Multiple Comparisons Test.

FIGS. 4A-4E. Encapsulation protects from degradation by RNAse A. FIG. 4A. Schematic of RNAse-containing conditions. RNAse A degrades the riboswitch (i) in bulk conditions and (ii) when co-encapsulated with reactants but is unable to reach reactants contained within vesicles (iii). FIG. 4B. Riboswitch response to NaF in bulk conditions with and without RNAse A added to reaction. FIG. 4C. Riboswitch activity as indicated by GFP/OA647 fluorescence when encapsulated with 3 mM NaF compared to the co-encapsulation of both 3 mM NaF and RNAse A. FIG. 4D. Response of encapsulated riboswitch to externally added NaF with RNAse A present in external solution. Black lines indicate mean and standard deviation. FIG. 4E. Histogram of data in FIG. 4D. Data plotted with lowless curve fitting. **** p≤0.0001, nonsignificant (ns) p>0.05; p-values generated using a One-Way ANOVA and Tukey's Multiple Comparisons Test.

FIGS. 5A-5E. Colorimetric sensors detect fluoride at environmentally relevant concentrations and in real-world samples. FIG. 5A. Schematic of encapsulated enzymatic readout. Riboswitch activation inside vesicles leads to the expression of C23DO, resulting in the production of a yellow product that is localized to the vesicle interior. FIG. 5B. Absorbance over time in populations of vesicles encapsulating a catecholase-based readout in response to external NaF. n=2 technical replicates. FIG. 5C. Absorbance as a function of external NaF concentration at two timepoints, t=2 hours and t=5 hours since initiating vesicle exposure to fluoride. n=2 technical replicates. FIG. 5D. Changes in absorbance over time inside of populations of vesicles incubated in samples derived from MilliQ water, tap water and lake water supplemented with either 0 mM NaF or 3 mM NaF. n=2 independent vesicle preparations. FIG. 5E. Colorimetric changes in vesicles as viewed through a microscope eyepiece and by eye in Eppendorf tubes.

FIG. 6. Microscopy timepoints of GFP expression inside vesicle sensors. Vesicles expressing the riboswitch construct encoding for GFP with 5 mM external NaF were assessed at four timepoints, t=0, 2, 6 and 24 hours. GFP signal was observed to be maximum at t=6 hours, consistent with other similar studies (30, 42), which was chosen as the timepoint at which future microscopy studies were assessed.

FIG. 7. OA647 retention in 2:1 cholesterol:POPC vesicles following encapsulation and protein expression. Vesicle populations exposed to increasing fluoride in the external solution exhibit the retention of a volume marker, OA647, even when RNAse A is present externally. While average fluorescence varies slightly between populations, corresponding to differences in the size of analyzed vesicles between conditions, all samples exhibit similar fluorescence profiles consistent with the retention of protein-sized molecules within the vesicle interior.

FIGS. 8A-8C. An encapsulated riboswitch responds specifically to fluoride. FIG. 8A. Fluoride permeates the vesicle membrane to initiate the expression of a GFP reporter inside vesicles. FIG. 8B. The external addition of NaCl does not result in robust GFP expression inside vesicles. FIG. 8C. GPF/OA647 fluorescence in vesicles with either NaF or NaCl added to the external buffer. While a response is observed to increasing chloride, the magnitude is significantly less than the response to fluoride and there is no observed population shift towards highly active vesicles. Differences in expression between fluoride and chloride containing conditions were clearly distinguishable, indicating sufficient specificity to fluoride over chloride. ****p £0.0001, nonsignificant (ns) p>0.05; p-values generated using a One-Way ANOVA and Tukey's Multiple Comparisons Test.

FIG. 9. pH decreases as increasing NaF is added externally to vesicles. Lipid/cholesterol vesicles containing HPTS dye without cell-free expression systems show changes in fluorescence after addition of anions to the external solution, indicating a cross-membrane effect on pH caused by externally added NaF. Compared to NaCl and buffer only controls, the pH of the vesicle interior decreases in the presence of externally added NaF, as indicated by a decreasing fluorescence ratio of HPTS, a pH-sensitive dye. These results indicate that fluoride ions may permeate the membrane as HF, bringing H⁺ ions with them as they pass through the membrane.

FIG. 10. Glycerol addition increases membrane permeability. Lipid/cholesterol vesicles encapsulating calcein, a self-quenching fluorescent dye, show slightly increasing cargo leakage following glycerol addition to the surrounding buffer. In both the presence and absence of RNAse, addition of increasing volumes of 0.02% glycerol solutions (1.25 mL of which was added in vesicle studies) leads to slightly higher levels of calcein dye release from the vesicle interior, indicating increased membrane permeability to small molecules.

FIGS. 11A-11B. Catecholase conversion in response to fluoride in bulk and inside of vesicles. FIG. 11A. Bulk reactions show slightly higher responses to 3 mM NaF supplemented in water samples taken from Lake and Tap water compared to laboratory-grade MilliQ water. Absorbance was also observed to increase in unsupplemented tap water, likely due to low levels of fluoride added to public drinking supply. FIG. 11B. In vesicles, absorbance increases specifically in the presence of supplemented NaF. Quantification is difficult, with 1 mM NaF exhibiting a slightly delayed response compared to 3 mM NaF but a similar expression profile and maximum absorbance. Responses are similar between all 3 mM samples regardless of water source, with a slight increase in expression in unsupplemented tap water at later timepoints (consistent with bulk data). (n=1 example vesicle preparation shown here).

FIGS. 12A-12C. Dose response in colorimetric vesicle sensors as a function of time-to-detection. FIG. 12A. Changes in absorbance in populations of vesicles exposed to increasing external NaF concentrations ranging from 0-5 mM. FIG. 12B. Sensor responses to increasing fluoride concentrations below 1 mM NaF. Beyond 2 hours, sensors can serve as a binary indicator of fluoride concentrations above the EPA Maximum Containment Level of 0.2 mM. FIG. 12C. Changes in absorbance over the course of five hourly timepoints in vesicles exposed to increasing concentrations of externally added fluoride. Significance assessed compared to the 0 mM condition. ns p>0.05, * p≤0.05, ** p≤0.01, *** p≤0.001, **** p≤0.0001; p-values generated using a Two-Way ANOVA and Dunnett Multiple Comparisons Test.

FIG. 13. GFP sensor responses to fluoride in real-world samples. GFP/OA647 fluorescence ratios observed in vesicles containing a GFP-based readout after incubation in outer solutions from laboratory grade water (MilliQ), tap water, and lake water supplemented with NaF. **** p≤0.0001, p-value generated using a One-Way ANOVA and Tukey's Multiple Comparisons Test.

### DETAILED DESCRIPTION

Cell-free systems have enabled the development of genetically encoded biosensors to detect a range of environmental and biological targets. Encapsulation of these systems in synthetic membranes to form artificial cells can reintroduce features of the cellular membrane, including molecular containment and selective permeability, to modulate cell-free sensing capabilities. Here, the inventors demonstrate robust and tunable performance of a transcriptionally regulated, cell-free riboswitch encapsulated in lipid membranes, allowing detection of membrane-permeable analytes.

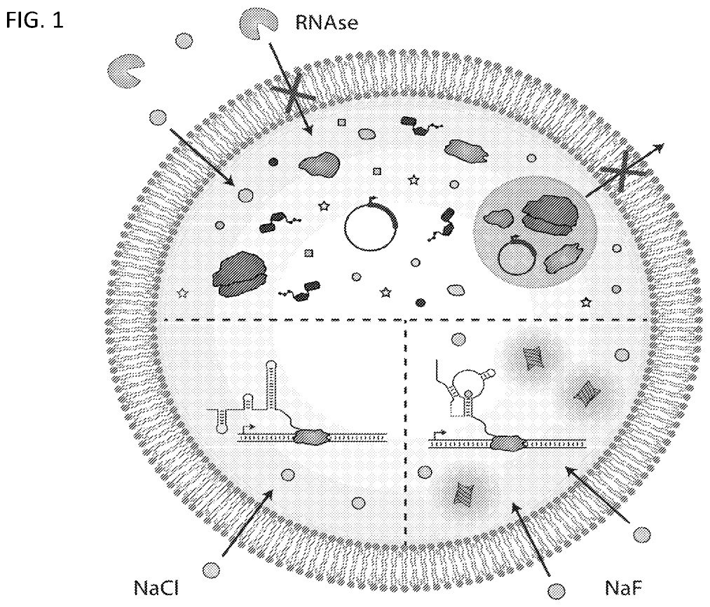

This disclosure provides artificial cell-based sensors by encapsulating a transcriptionally regulated, analyte-responsive riboswitch and a cell-free system within bilayer membranes (FIG. 1). The inventors first encapsulate the riboswitch, then demonstrate its ability to detect externally added membrane-permeable analytes and show that membrane composition can be modified to tune sensitivity to the membrane-permeable analytes. The inventors also demonstrate that encapsulation protects cell-free reactions from sample degradation, particularly from extravesicular degradative enzymes. Finally, the inventors couple riboswitch output to both fluorescent and colorimetric reporters and show that vesicle-based sensors can detect membrane-permeable analytes. The Examples demonstrate the detection of fluoride with a fluoride-responsive riboswitch at environmentally relevant concentrations and in real-world water samples. This work demonstrates the potential of encapsulated, riboswitch-based sensors for biosensing applications, complimenting existing cell-free sensor engineering strategies and enabling sensing in otherwise inhospitable environments.

### Compositions

In a first aspect, provided herein is a composition comprising a cell-free system and a construct encoding a riboswitch, wherein the riboswitch is responsive to an analyte, wherein the riboswitch is coupled to a reporter protein, and wherein the composition is encapsulated within a membrane. When the composition is exposed to an analyte, the riboswitch is activated and the reporter protein is transcribed.

A “cell-free system”, as described herein, includes a crude or partially-purified cell extract, and a suitable reaction buffer for promoting cell-free protein synthesis from a DNA expression template. The DNA expression template typically encodes an open reading frame operably linked to a promoter element for a DNA-dependent RNA polymerase. Therefore, the cell-free system may also include a DNA-dependent RNA polymerase to direct transcription of an RNA translation template encoding the open reading frame. The cell-free system may further include NTPs, amino acids, buffers, energy sources, etc. needed for transcription and translation of the DNA template. In exemplary embodiments, the cell-free reaction comprises a cell extract, a reaction buffer containing the small molecules required for transcription and translation (NTPs, amino acids, buffering salts, crowding agents, and an energy source), to which DNA templates and inducers are added at a ratio of approximately 30/30/40, as shown in Table 4 below. A reaction mixture is considered complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The term “transcription factor” refers to a protein that regulates transcription of another protein, typically by interacting with one or more cis-acting DNA sequence in or near the promoter for the other protein. A transcription factor may increase expression or decrease expression depending upon whether the transcription factor is activated or deactivated. A transcription factor may become activated or deactivated by an interaction with another molecule (e.g., a target molecule as described above).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as a “gene product.” If the polynucleotide is derived from genomic DNA, expression may include splicing of the mRNA in a eukaryotic cell.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, bacteriophage polymerases such as, but not limited to, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, the term “crude” refers to a cell extract comprising components obtained by disrupting and lysing cells and, at best, minimally purifying components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

A variety of methods exist for preparing an extract competent for cell-free protein synthesis, including those disclosed in U.S. Patent Application Publication No. 2014/0295492 and U.S. Patent Application Publication No. 2016/0060301, the contents of which are incorporated by reference in their entireties. The cellular extract of the platform may be prepared from a cell culture of a prokaryote (e.g., E. coli). While E. coli is exemplified herein, the bacterial species is not intended to be limiting. Other bacterial species suitable for the compositions and methods disclosed herein include but are not limited to (e.g., Bacillus species such as Bacillus subtilis, Vibrio species such as Vibrio natrigens, Pseudomonas species, etc.). The cell culture may be in stationary phase. Stationary phase may be defined as the cell culture having an OD600 of greater than about 3.0, 3.5, 4.0, 4.5, 5.0, 5.5, 6.0, 6.5, 7.0, 7.5, 8.0, or having an OD600 within a range bounded by any of these values. Further methods for preparing a cell-free system are disclosed in International Patent Application Publication No. WO2020185451A2, the contents of which are incorporated by reference in its entirety. Cell-free protein synthesis (CFPS) has also been described in the art. (See, e.g., U.S. Pat. Nos. 6,548,276; 7,186,525; 8,734,856; 7,235,382; 7,273,615; 7,008,651; 6,994,986 U.S. Pat. Nos. 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties).

As described above, the cell-free system is able to synthesize a protein encoded by a DNA expression template. The DNA expression template used in the compositions described herein are constructs encoding a riboswitch coupled to a reporter protein. As used herein, the terms “construct” and “expression cassette” refer to a recombinant polynucleotide, i.e., a polynucleotide that was formed artificially by combining at least two polynucleotide components from different sources (natural or synthetic). For example, the constructs described herein comprise a transcriptionally regulated riboswitch and a reporter protein operably linked to a promoter. Constructs can be generated using conventional recombinant DNA methods. As used herein, the term “promoter” refers to a cis-acting DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. By way of example, but not by way of limitation, promoters useful in the compositions and methods disclosed herein include promoters from gram positive or gram negative bacteria, or synthetic derivatives thereof. The E. coli promoter BBa_J23119_Spe1, and the E. coli σ70 promoter are two non-limiting examples. A promoter is “operably linked” to a polynucleotide if the promoter is connected to the polynucleotide such that it may affect transcription of the polynucleotide. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Constructs and vectors contemplated herein may comprise a heterologous promoter (e.g., a prokaryotic or eukaryotic promoter) operably linked to a polynucleotide that encodes a protein. A “heterologous promoter” refers to a promoter that is not the native or endogenous promoter for the protein or RNA that is being expressed.

The term “vector” refers to a nucleic acid molecule capable of propagating another nucleic acid to which it is linked. The term includes the vector as a self-replicating nucleic acid structure as well as a vector incorporated into the genome of a host cell into which it has been introduced. The term vector encompasses “plasmids”, the most commonly used form of vector. Plasmids are circular double-stranded DNA loops into which additional DNA segments (e.g., those encoding peptides) may be ligated. In some embodiments, the vector is a mini-circle DNA (mcDNA) vector. Mini-circle DNA vectors are episomal DNA vectors that are produced as circular expression cassettes devoid of any bacterial plasmid DNA backbone. See, for example, System Biosciences, Mountain View CA, MN501A-1. Their smaller molecular size enables more efficient transfections and offers sustained expression over a period of weeks as compared to standard plasmid vectors that only work for a few days. The vectors may further a comprise heterologous nucleic acid backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of encoded peptides.

The disclosed methods and systems utilize one or more expression cassettes. In exemplary embodiments, the expression cassette comprises one or more of the following components: a promoter (e.g., a synthetic constitutive E. coli Promoter BBa_J23119_Spe1, to drive strong transcription and/or a T7 promoter); a fluoride-sensing riboswitch (e.g., the Bacillus cereus fluoride riboswitch sequence, which induces transcription based on the presence of fluoride); and an encoded reporter (e.g. the coding sequence for an enzyme such catechol 2,3-dehydrogenase or a fluorescent protein such as GFP or a derivative thereof). The cassette may also include a ribosome binding sequence (e.g., a synthetic ribosome binding site to drive strong translation) and a transcription terminator (e.g., the T1/TE double transcriptional terminator). An expression cassette comprising the one or more components may be utilized to sense fluoride in a cell-free gene expression reaction, wherein the reaction comprises metabolism from a host strain that provides energy; provides cofactor regeneration; provides enzymes used for cell-free sensing; or any combination thereof as well as exogenously supplied cell-free protein synthesis reagents and an RNA polymerase.

The term “riboswitch” refers to a cis-acting RNA element present in mRNA which binds a small molecule or analyte as an effector molecule, resulting in change in expression of the proteins encoded by the mRNA, either at the level of transcription or at the level of translation. Riboswitches may be divided into two parts which include an aptamer and an expression platform. The aptamer is formed by the mRNA after transcription. The aptamer then binds the effector molecule which results in structural changes in the expression platform that modulate expression of the protein encoded by the mRNA, either at the level of transcription or at the level of translation. A riboswitch thus controls expression of the protein encoded by the mRNA in response to the effector molecule. Riboswitches that are responsive to fluoride are known in the art. (See, e.g., U.S. Pat. No. 9,580,713, “Fluoride-Responsive Riboswitches, Fluoride Transporters, and Methods of Use”; the content of which is incorporated herein by reference in its entirety).

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. A polynucleotide may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present compositions and methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

As used herein the term “reporter” or “reporter protein” refers to a protein that can be detected in a reaction mixture, typically in response to the presence of an analyte in the reaction mixture. For example, a reporter may be expressed and detected in a sample when an analyte such as fluoride promotes expression of the reporter protein in the sample. In some embodiments, the reporter protein comprises an enzyme, and can be detected in a sample when contacted with an appropriate substrate. Numerous reporter proteins and reporter protein/substrate combinations are well known in the art. By way of example, but not by way of limitation, exemplary reporter proteins include fluorescent proteins, enzymes that create colored products, and luciferase. Non-limiting examples include Green Fluorescent Protein, Red Fluorescent Protein, Yellow Fluorescent Protein, catechol 2,3-dehydrogenase (C23DO), lacZ, derivatives thereof, and the like. In embodiments in which the reporter protein is a fluorescent protein, the composition may further comprise a volume marker. A “volume marker” refers to a protein-conjugated dye, which allows for detection of the interior of the vesicle. In exemplary embodiments, the reporter protein and the volume marker is ovalbumin-conjugated Alexafluor 647 (OA647). In embodiments in which the reporter protein is colorimetric enzyme, the composition may further comprise a substrate for the enzyme, wherein binding of the enzyme to the substrate results in emission of a color by the substrate. In exemplary embodiments, the colorimetric reporter protein/enzyme is catechol (2,3)-dioxygenase (C23DO), and the substrate is catechol.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

The term “analyte” refers to any molecule of interest in a test sample and may include so-called “small molecules” or metabolites of small molecules. In exemplary embodiments, the analyte is fluoride.

The compositions described herein are encapsulated within a membrane. As used herein, the term “encapsulate,” “encapsulated,” or “encapsulation” refers to enclosing or compartmentalizing a composition within a membrane. The membrane provides a semi-permeable barrier between the contents encapsulated within the membrane and the external environment.

The membrane encapsulating the compositions disclosed herein may comprise a lipid membrane. A “lipid membrane,” “lipid structure,” or “lipid layer” means a continuous, self-assembled barrier comprising a plurality of amphiphilic lipids. The lipid layer may comprise a single layer of amphiphilic lipids, e.g., a micelle or a reverse micelle, having a hydrophilic surface and a hydrophobic surface. In exemplary embodiments, the membrane is a lipid bilayer comprising two layers of amphiphilic lipids having an inner-hydrophilic surface, an outer-hydrophilic surface, and a hydrophobic core disposed between the inner-hydrophilic surface and the outer-hydrophilic surface, e.g., a liposome, a lipid nanoparticle, a cell, a cellular organelle, or a 2-dimensional membrane.

“Amphiphilic lipid” means any chemical compound having both hydrophilic and hydrophobic properties and typically composed of a polar head group and lipophilic tail. The polar head group may charged or uncharged. Suitably, the polar head groups may comprise anionic head groups (such as carboxylates, sulfates, sulfonates, or phosphates), cationic head groups (such as ammoniums), or uncharged head groups (such as alcohols). The lipophilic tail is typically a saturated or unsaturated alkyl or a saturated or unsaturated alkylene having at least four carbon atoms, suitably between 6 and 24 carbon atoms. Exemplary amphiphilic lipids include, without limitation, phospholipids (e.g., sphingomyelins or phosphoglycerides such as phosphatidylserines, phosphatidylethanolamines, phosphatidylinositols, or phosphatidylcholines), glycolipids, fatty acids, amphiphilic di-block copolymers, amphiphilic tri-block copolymers, amphiphilic dendrimers, amphiphilic dendrons, or peptide amphiphiles.

In exemplary embodiments, the membrane comprises 1-palmitoyl-2-oleoyl-glycero-3-phosphocholine (POPC). The membrane may further comprise at least one of cholesterol, oleic acid, and poly(ethylene oxide)-b-poly(butadiene) (PEO-b-PBD) diblock copolymer. In exemplary embodiments, the membrane comprises a 2:1 ratio of cholesterol:POPC.

The membranes encapsulating the compositions described herein may form vesicles. A “vesicle” refers to any closed-structure comprising a lipid layer enclosing a liquid or gas. Vesicles may vary in size from about 10 nm to about 100 μm in diameter. In some cases, the vesicles may be characterized as “small” (typically less than 100 nm in diameter), “large” (typically 100 nm to 1 μm), or “giant” (typically greater than 1 μm). Vesicles may be unilamellar or multilamellar. Exemplary vesicles include, without limitation, micelles, reverse micelles, small unilamellar liposome vesicles (SUVs), large unilamellar liposome vesicles (LUVs), giant unilamellar liposome vesicles (GUVs), cells, organelles, vacuoles, lysosomes, transport vesicles, secretory vesicles, exosomes, microvesicles, membrane particles, apoptotic blebs, polymersomes, dendrimersomes, peptide-amphiphile vesicles, gas vesicles, and synthetically made vesicles.

The lipid membrane may comprise additional components, such as a protein, a carbohydrate, a sterol, or any combination thereof. Proteins may include surface proteins, integral proteins, transmembrane proteins, globular proteins, glycoproteins, and oligopeptides.

### Methods

In a second aspect, provided herein is a method of detecting an analyte in a sample, the method comprising contacting the sample with a composition comprising a cell-free system and a construct encoding a riboswitch coupled to a reporter protein, wherein the composition is encapsulated within a membrane; and detecting a signal emitted by the reporter protein; wherein the riboswitch is responsive to the analyte; and wherein if the analyte is present in the sample, the signal is emitted. The composition used in the method may comprise any of the compositions described herein.

As used herein, the term “sample” refers to a small or representative part or quantity from a whole or group. A sample may be in liquid, solid or gaseous form, and may or may not include an analyte, e.g., fluoride. In embodiments, samples are environmental samples (e.g., removed from the environment), industrial samples (e.g., removed from a factory or industrial setting or from a region of factory, industrial, or commercial outflow), or biological samples (e.g., taken from an animal or plant). By way of example, but not by way of limitation, exemplary liquid samples include water (e.g., from ponds, springs, lakes, creeks, municipal sources, etc.), commercial waste effluent, and blood products. In some embodiments the biosensors and methods provided herein are used in water safety applications, such as screening municipal or environmental water samples for the presence and/or concentrations of fluoride or other toxins. In other embodiments, the sample is a biological sample obtained from an individual (e.g., a human subject, a non-human mammal). The sample is, in some cases, a diagnostic sample. The sample type will vary depending on the target analyte. For example, diagnostic samples can be a serum sample, blood sample, sputum sample, urine sample, or other biological fluid. In some cases, serum samples have been frozen (e.g., at −80° C.) prior to testing. In some cases, samples appropriate for use according to the methods provided herein are “non-biological” in whole or in part. Non-biological samples include, without limitation, plastic and packaging materials, paper, clothing fibers, and metal surfaces. In some embodiments, solid samples may be treated with a liquid (e.g., dissolved or soaked in a liquid such as water or other solvent); likewise a gaseous sample may be exposed to or treated with a liquid prior to testing via the compositions and methods disclosed herein.

The term “detect” or “detection” as used herein indicates the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest, e.g. a reporter protein encoded by the construct or substrate bound by the reporter protein.

### Kits

In a third aspect, provided herein is a kit comprising any of the compositions described herein, wherein the compositions are provided in a container. In preferred embodiments, the container is large enough that a sample may be added to it. Any suitable container may be used, e.g. a reaction tube, or a vial. In embodiments, the container is clear and any colorimetric signal emitted is visible to the naked eye and may be observed. The kit may further include buffers and reagents necessary for detecting a signal emitted when the analyte is present in the sample. The kit may further include a dropper for dispensing controlled volumes of liquid, such as a controlled volume disposable Pasteur pipette. The kit may further include a written insert component comprising instructions for carrying out the methods described herein.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—Robust and Tunable Performance of a Cell-Free Biosensor Encapsulated in Lipid Vesicles

### a Transcriptionally Regulated Fluoride Riboswitch can Function Inside Lipid Vesicles

The inventors first sought to confirm that a transcriptional riboswitch can function when encapsulated inside lipid vesicles. For the riboswitch, the inventors chose the fluoride responsive riboswitch from Bacillus cereus, which was characterized previously (3). In earlier studies, the inventors showed that this riboswitch can be used to control the expression of several different reporter proteins and fluorescent RNA aptamers in bulk E. coli extract-based cell-free systems (3). In this system, the fluoride riboswitch is encoded within a single DNA template, downstream of a consensus E. coli promoter sequence, and upstream of a reporter coding sequence. In the absence of fluoride, E. coli polymerase transcribes the riboswitch sequence, causing it to fold into a conformation that exposes a transcriptional terminator hairpin and subsequently causes RNA polymerase to stop transcription (38). In the presence of fluoride, fluoride binding to the riboswitch aptamer domain prevents the terminator from folding, allowing transcriptional elongation of the reporter coding sequence.

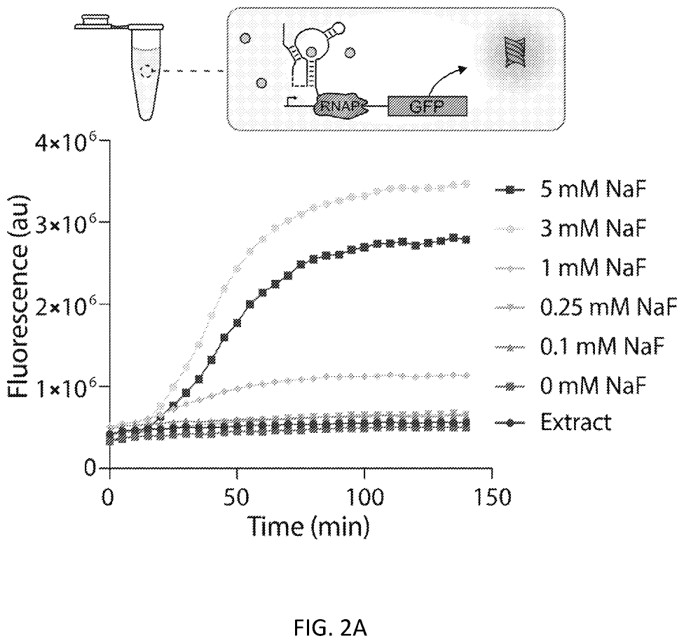

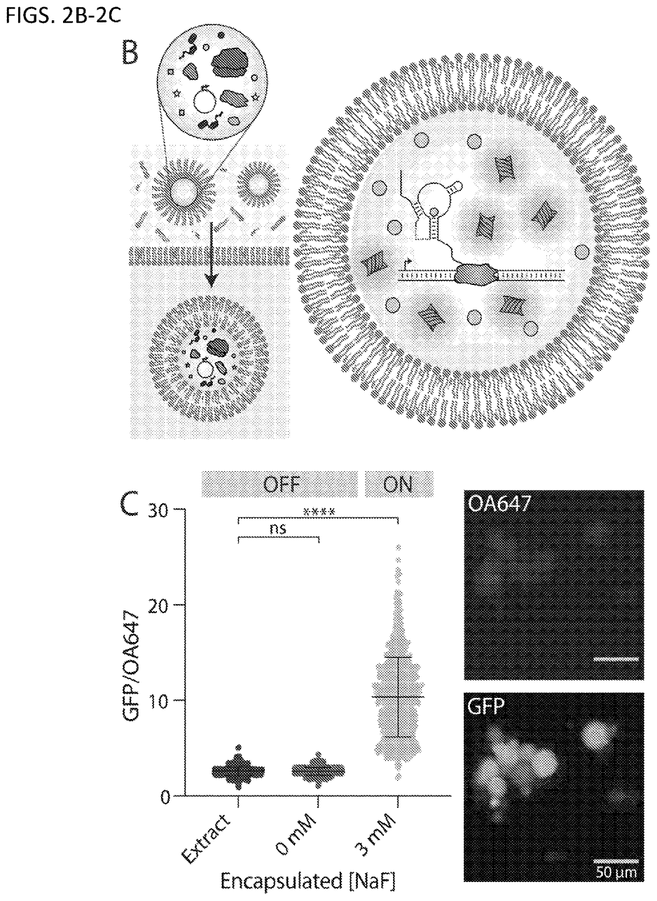

In this study, the inventors first chose to use a super folder green fluorescent protein (GFP) reporter, as it allows convenient measurement of riboswitch activity. For the cell-free system the inventors used an E. coli S30 lysate prepared with runoff and dialysis, which has been shown to allow the function of biosensors that require bacterial polymerases (39). Embedding the riboswitch DNA template into the extract system alongside varying concentrations of sodium fluoride (NaF) showed, as expected (3), an increase in GFP fluorescence as fluoride concentrations increased up to 3 mM, followed by a decrease in fluorescence at higher concentrations (FIG. 2A). This decrease is likely caused by fluoride inhibition of the gene expression machinery (40) and is consistent with the inventors' previous characterization of this construct (3). Accordingly, the inventors used 3 mM NaF for the remainder of this study to obtain the expected maximum fluorescent output of the system.

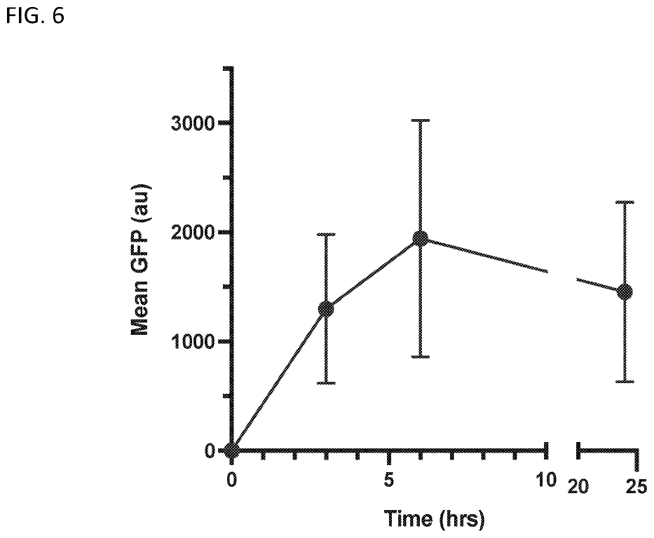

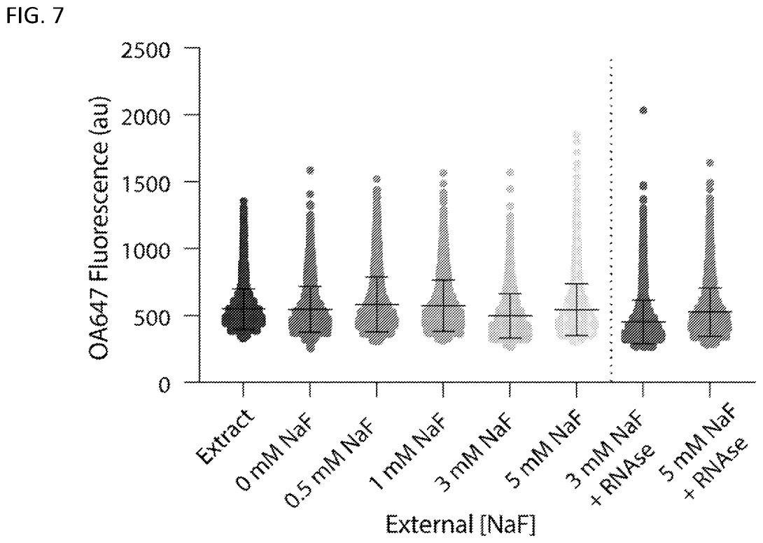

The inventors then set out to assess whether the fluoride riboswitch could retain functionality when encapsulated within lipid vesicles. Vesicles were synthesized using a water-in-oil emulsion transfer method (FIG. 2B) (41). In this method, various membrane amphiphiles (e.g. lipids, cholesterol, fatty acids, diblock copolymers) are dissolved into an oil phase and an emulsion is formed by vortexing the aqueous cell-free reaction into this mixture. The emulsion is then layered onto a second aqueous layer containing the small-molecule components of the cell-free reaction, and emulsified droplets are centrifuged through the oil-water interface to generate unilamellar vesicles. Previous characterization of vesicles formed using this method has yielded encapsulation efficiencies of ~98%, indicating high levels of entrapment of aqueous components (41). Vesicle synthesis using this approach yields a distribution of different vesicle sizes on the 5-50 μm scale, which could impact the quantification of fluorescence (42). To control for this, the inventors also incorporated a protein-conjugated dye, ovalbumin-conjugated Alexafluor 647 (OA647), which served as a volume marker and allowed them to detect the vesicle interior regardless of GFP expression level (30, 43). After synthesis, vesicles were incubated under varying conditions at 37° C., and protein expression was assessed using epifluorescent microscopy after 6 hours of incubation (FIGS. 2C and 6) (30, 42). Vesicles were imaged using GFP and Cy5.5 channels, and images were analyzed using the NIS-elements AR software program (44), which allowed the inventors to automatically select vesicle interiors using the OA647 marker and report GFP fluorescence in those regions. This protocol allowed the inventors to analyze hundreds of vesicles per sample, maintain the same selection parameters between samples, and minimize the impact of user selection bias in the analysis. Additionally, the encapsulated volume marker allowed the inventors to report GFP expression relative to OA647 fluorescence to control for possible variability in vesicle size or loading. Finally, to control for variations in levels of extract autofluorescence between vesicle preparations, all samples compared to an “extract only” control population were analyzed for each preparation. Using these methods, the inventors were able to ensure that the measurements were isolated to intact (non-lysed) vesicles which retained their protein cargo (FIG. 7).

Using the above approach, the inventors encapsulated cell-free reactions with and without fluoride present in the bulk reaction mixture. They chose to use a 2:1 ratio of cholesterol and POPC phospholipid as membrane amphiphiles due to their previous use in similar encapsulated expression studies (21, 28-30, 43). Upon co-encapsulation of the riboswitch with 3 mM NaF GFP expression inside vesicles was observed, indicating the riboswitch was in the “ON” state (FIG. 2C). In contrast, in the absence of DNA (extract only) or in the absence of fluoride (0 mM NaF) minimal GFP expression was observed, indicating an “OFF” state (FIG. 2C). This high level of GFP induction inside vesicles by fluoride indicates that membrane encapsulation does not eliminate the ability of the riboswitch to fold properly and does not cause significant nonspecific expression.

The inventors observed that populations of vesicles exhibited variations in GFP fluorescence between individual liposomes after 6 hours of incubation (FIG. 2C), a phenomenon which has been observed in similar studies across multiple encapsulation protocols (21, 24, 30, 42, 45-47). It has been hypothesized that these variations in gene expression may be caused by variability in vesicle loading and/or varied levels of molecular exchange with the surrounding buffer for vesicles of different sizes (24, 45, 46, 48). To report this variability across vesicle populations metrics of skew for each population result have been included (Tables 1-3). Even after taking this variability into account, however, induction of GFP expression is clearly observable across the vesicle population, indicating proper riboswitch sensor activity and a robust response to fluoride in encapsulated sensors.

### External Fluoride can be Detected by an Encapsulated Riboswitch

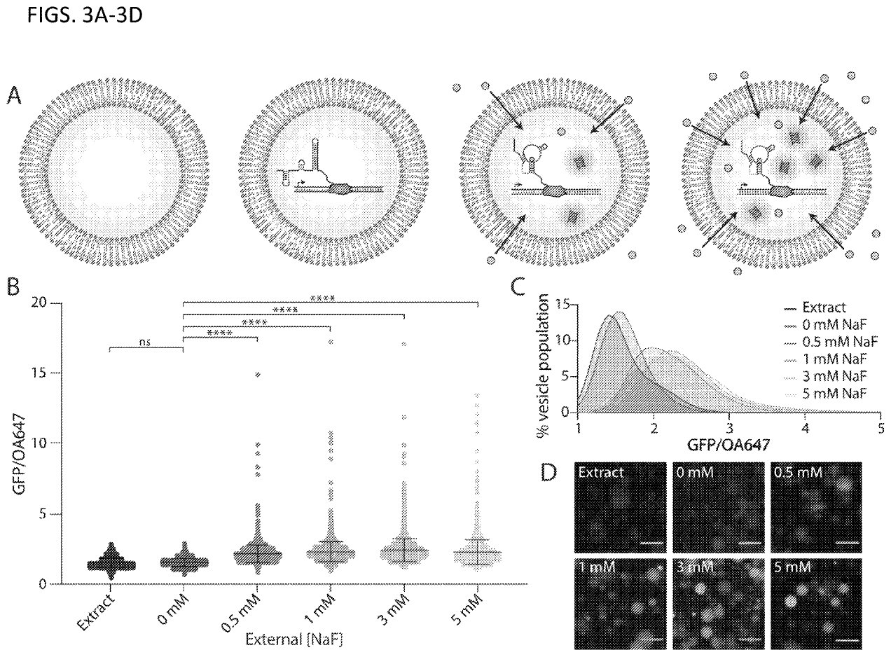

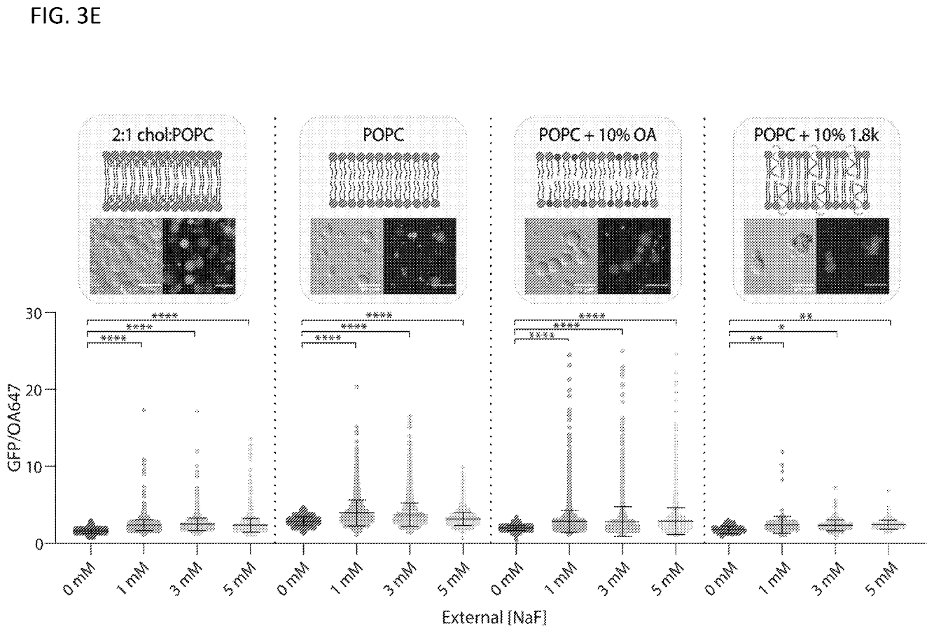

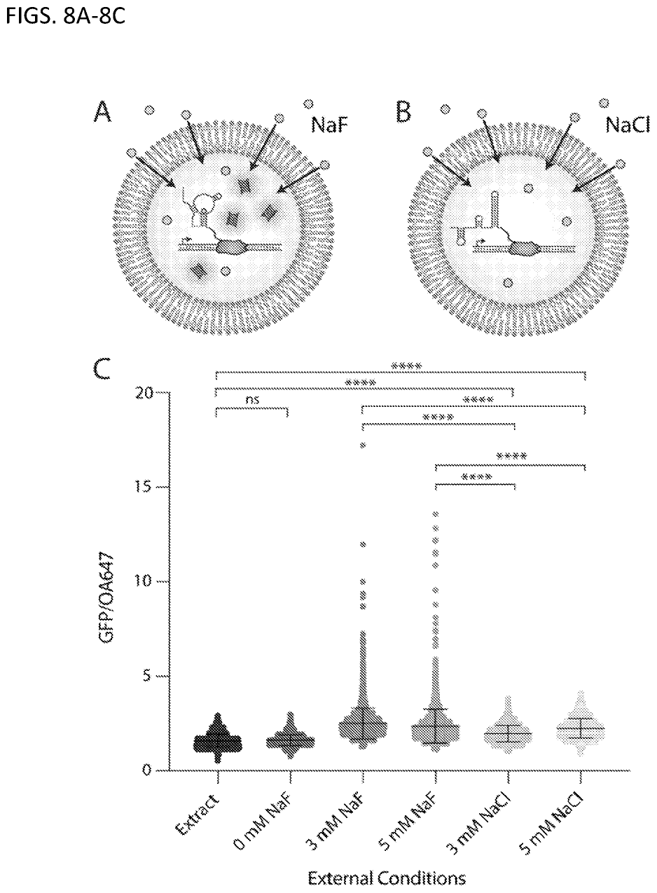

The inventors next sought to determine whether the encapsulated riboswitch could detect fluoride added to the external solution of pre-assembled sensor vesicles. To assess this, vesicles containing cell-free reactions without NaF present in the reaction mixture were prepared. Then NaF was titrated into the solution surrounding vesicles (FIG. 3A) and vesicles were imaged following incubation for 6 hours at 37° C. Increasing GFP expression with increasing concentrations of NaF up to 3 mM and a slight decrease in average fluorescence at 5 mM, consistent with bulk studies (FIGS. 3B-3D) was observed. Vesicle populations exhibited increases in both mean GFP/OA647 fluorescence and population skew in response to increasing fluoride, either of which could serve as a metric of fluoride detection (Table 1). All fluoride-containing conditions exhibited a significant increase in fluorescence compared to no-DNA and no-fluoride controls (FIGS. 3B-3C, Table 1). When incubated with chloride, a similarly monovalent anion, a slight response to increasing ion concentration was observed, however these responses were significantly lower than any response to fluoride and did not exhibit any of the highly active vesicles that were observed in all fluoride-containing conditions (FIG. 8). These responses were easily distinguishable between fluoride and chloride, indicating sufficient specificity to fluoride, as has been observed previously (3). Taken together, these results indicate that increasing concentrations of fluoride added to the extravesicular environment can be detected by the encapsulated riboswitch.

TABLE 1
GFP expression in response to externally added NaF in 2:1
cholesterol:POPC vesicles. Descriptive statistics of GFP
expression in populations of vesicles exposed to increasing
amounts of externally added NaF. Statistical analysis
was computed compared to 0 mM NaF conditions.

| | | Mean | | Significantly | | |
|---|---|---|---|---|---|---|
| External NaF | | Fluorescence | | different | Skew- | |
| concentration | | GFP/OA647 | SEM | than 0 mM? | ness | Kurtosis |
| Extract | | 1.596 | 0.0054 | ns | 0.5225 | 0.8674 |
| 0 | mM | 1.625 | 0.0062 | | 0.4007 | 0.4155 |
| 0.5 | mM | 2.250 | 0.0126 | **** | 2.205 | 13.12 |
| 1 | mM | 2.388 | 0.0143 | **** | 1.824 | 9.110 |
| 3 | mM | 2.500 | 0.0167 | **** | 2.083 | 8.960 |
| 5 | mM | 2.372 | 0.0166 | **** | 4.587 | 46.19 |

**** p £ 0.0001, nonsignificant (ns) p > 0.05; p-values generated using a One-Way ANOVA and Tukey's Multiple Comparisons Test.

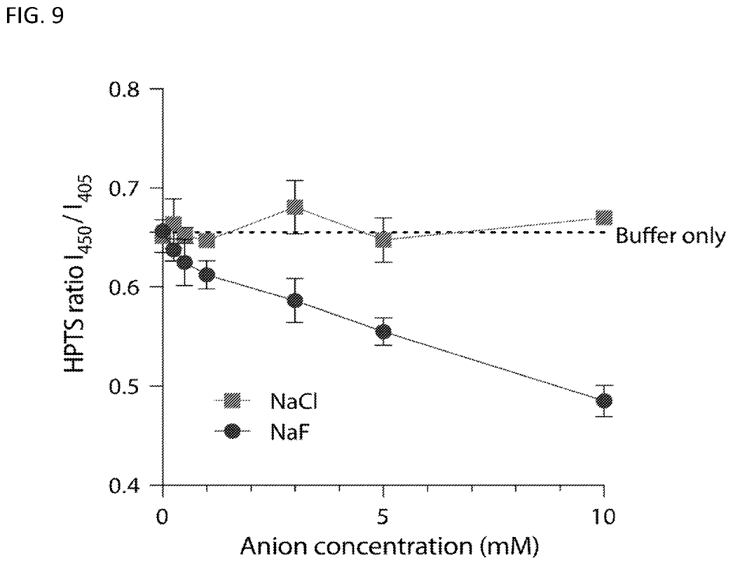

This result was somewhat unexpected, as it was anticipated that the membrane would be relatively impermeable to charged fluoride ions. The observed magnitude of fluoride permeability may be explained in part by the transient formation of hydrofluoric acid (HF). HF has been shown to exhibit a permeability coefficient that is seven orders of magnitude greater than fluoride anions through lipid/cholesterol bilayers, indicating that HF travels through the membrane much more readily than its anionic F-counterpart (40, 49). The inventors confirmed this effect by encapsulating a pH sensitive dye, HPTS, which reported a slight decrease in pH in the vesicle interior upon the addition of fluoride to the external buffer (FIG. 9). This result indicates an increase in proton concentration inside the vesicle as fluoride concentration increases, consistent with cross-membrane transport of HF.

As it was observed that fluoride could pass through the membrane to interact with the encapsulated riboswitch, the inventors wondered if they could alter the composition of vesicle membranes to modulate membrane permeability and thereby modulate sensitivity of these sensors to external fluoride. Membrane permeability to small molecules depends significantly on membrane composition, as various lipid chain chemistries and contributions from other amphiphilic components can impart an effect on membrane physical properties. Cholesterol, a major component of the original 2:1 cholesterol:POPC lipid composition, is known to decrease membrane permeability by increasing lipid packing and altering membrane fluidity and rigidity (50). PEO-b-PBD diblock copolymers are similarly known to reduce membrane permeability by increasing membrane viscosity, introducing steric barriers from the polyethylene glycol groups that assemble at the membrane interface, and increasing thickness and chain entanglements within the hydrophobic portions of the membrane (51, 52). In contrast, fatty acids such as oleic acid have been shown to increase membrane permeability to ionic solutes by incorporating single hydrocarbon chains that have a different shape and amphipathicity than diacyl chains, reducing lipid chain packing and enhancing fluidity of the bilayer (53, 54). Using this series of amphiphilic molecules, the capacity of membrane amphiphiles and the resulting membrane permeability to modulate the performance of an encapsulated cell-free sensor was assessed.

To explore the effect of these amphiphiles on membrane permeability to fluoride, vesicles were prepared with either 1) pure POPC lipid, 2) POPC lipid+10% oleic acid (OA), or 3) POPC lipid+10% PEO₁₄-b-PBD₂₂ diblock copolymer (MW=1.8 kDa, hereafter referred to as 1.8k) components in the lipid/oil mixture, encapsulating cell-free reactions as normal (FIG. 3E). The inventors observed an increase in overall GFP expression in both pure POPC lipid and POPC+10% OA conditions compared to the original 2:1 cholesterol:POPC lipid composition (for 3 mM external fluoride, 2:1 cholesterol:POPC vs. POPC: p<0.0001; 2:1 cholesterol:POPC vs. 10% OA: p<0.0001). These results are consistent with the removal of cholesterol and the addition of oleic acid, respectively, both of which are expected to increase membrane permeability. In addition, the inclusion of oleic acid in vesicle membranes led to a reduction of sensor dynamic range, measured via a reduced concentration dependence of GFP expression on NaF concentration (10% OA+1 mM NaF vs. 10% OA+3 mM NaF: p>0.9999; 10% OA+1 mM NaF vs. 10% OA+5 mM NaF: p=0.8149). In contrast, vesicles containing 10% 1.8k diblock copolymer exhibited reduced responsiveness to increasing external NaF, indicating reduced membrane permeability. Mean POPC vesicle fluorescence peaked at 1 mM NaF, while 10% 1.8k diblock copolymer responses were maximum at 5 mM NaF (Table 2). All fluoride-containing conditions for samples containing 10% OA were statistically indistinguishable. Taken together, these results indicate that exchanging membrane components to control membrane permeability provides a handle to tune the sensitivity of an encapsulated riboswitch to an analyte of interest. Further, the selection of highly permeable amphiphiles does not necessarily improve sensor performance and may instead increase overall signal but limit sensor resolution. A balance between analyte access and desired sensing behavior is likely an important consideration for engineering encapsulated biosensing systems depending on the desired application.

TABLE 2
GFP expression in response to externally added NaF in vesicles
with varying membrane compositions. Descriptive statistics of
GFP expression in populations of vesicles with varying membrane
compositions in response to increasing concentrations of externally
added NaF. Statistical analysis was computed compared to 0 mM
NaF conditions for each respective membrane composition.

| | Mean | | | | |
|---|---|---|---|---|---|
| External NaF | GFP/ | | Different | Skew- | Kur- |
| concentration | OA647 | SEM | than 0 mM? | ness | tosis |
| POPC, 0 mM | 2.904 | 0.01074 | | 0.1471 | −0.3680 |
| POPC, 1 mM | 3.968 | 0.02997 | **** | 2.063 | 7.183 |
| POPC, 3 mM | 3.724 | 0.02570 | **** | 2.433 | 9.854 |
| POPC, 5 mM | 3.190 | 0.01478 | **** | 1.528 | 5.072 |
| 10% OA, 0 mM | 1.997 | 0.004865 | | 0.3135 | 0.1434 |
| 10% OA, 1 mM | 2.850 | 0.02232 | **** | 6.120 | 59.59 |
| 10% OA, 3 mM | 2.839 | 0.03147 | **** | 4.848 | 32.92 |
| 10% OA, 5 mM | 2.909 | 0.03352 | **** | 5.038 | 38.90 |
| 10% 1.8k, 0 mM | 1.807 | 0.05333 | | 0.6967 | −0.2151 |
| 10% 1.8k, 1 mM | 2.414 | 0.07304 | ** | 5.698 | 39.98 |
| 10% 1.8k, 3 mM | 2.353 | 0.04787 | * | 2.199 | 11.16 |
| 10% 1.8k, 5 mM | 2.458 | 0.04654 | ** | 3.247 | 19.66 |

**** p £ 0.0001,
** p £ 0.01,
* p £ 0.05, nonsignificant (ns) p > 0.05; p-values generated using a One-Way ANOVA and Tukey's Multiple Comparisons Test.

Encapsulation Protects Sensor Components from Degradation

Having established that these vesicle sensors can detect external fluoride, how they might function in complex samples was explored next. One major benefit of membrane encapsulation is the ability to leverage the semipermeable barrier formed by the membrane to contain and protect encapsulated components. Cell-free reactions, particularly those using riboswitches, are highly sensitive to the presence of nucleases and proteases which can degrade sensor components before a target analyte is encountered (19). Due to their large size, however, enzymes are unable to pass through the vesicle membrane to access encapsulated reactants.

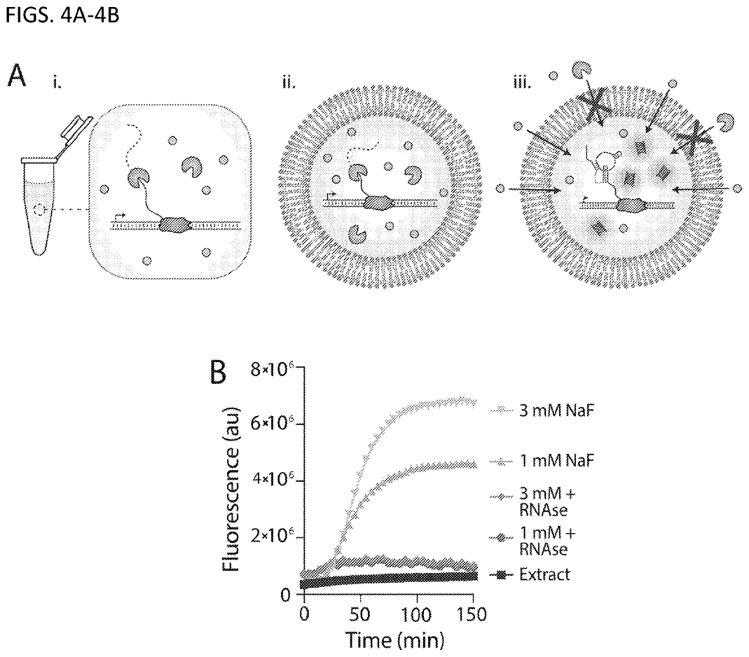

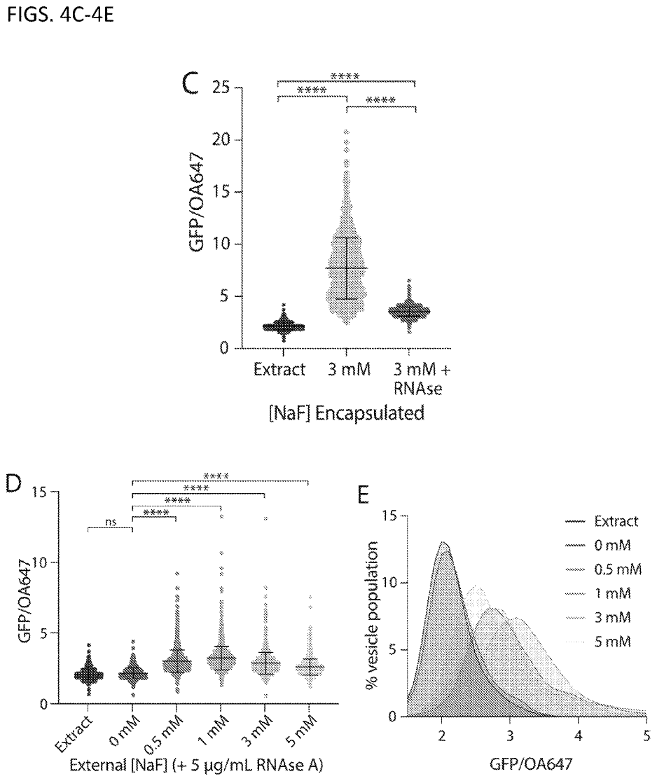

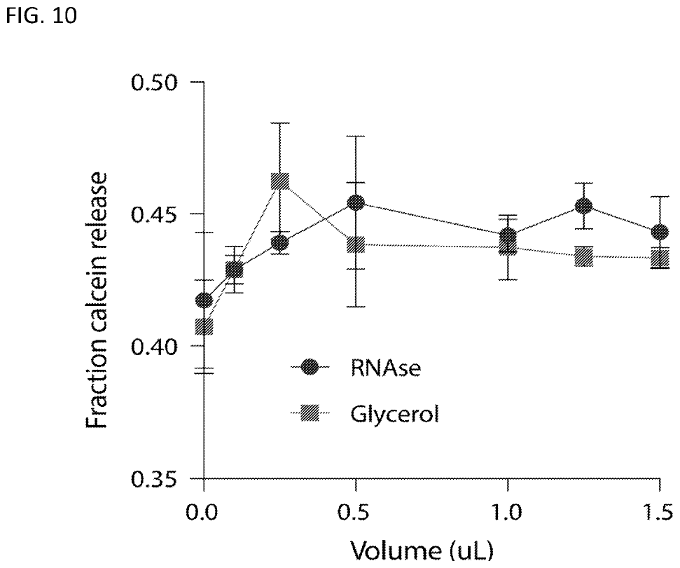

To determine whether the vesicle membrane can sufficiently protect encapsulated reactions from external degradation, the inventors tested various vesicle assemblies in the presence of RNAse A (FIG. 4A). It was observed that RNAse completely eliminated the riboswitch response to NaF both in bulk conditions and when RNAse was co-encapsulated with the cell-free reaction inside vesicles (FIGS. 4B-4C). In contrast, encapsulated sensors maintained the ability to respond to externally added NaF when RNAse was present in the external sample (FIGS. 4D-4E, Table 3). Interestingly, a decrease in mean GFP fluorescence at higher external NaF concentrations was noticed-a trend which was not observed in non-RNAse exposed samples. It was hypothesized that this difference was due to slightly higher degrees of membrane permeability from the addition of small amounts of glycerol in the RNAse buffer (FIG. 10), which could cause increased reaction poisoning with high fluoride concentrations. In addition, glycerol has been demonstrated to inhibit cell-free protein synthesis (CFPS) at high concentrations, and may be causing slight effects even at the low concentrations used here (55). Nevertheless, all vesicle populations exhibited increased GFP expression in the presence of fluoride, demonstrating simultaneous permeation of fluoride into the vesicle interior and exclusion of RNAse A from the cell-free reaction. These results indicate that encapsulation in bilayer membranes can sufficiently exclude RNAse from the reaction environment, thereby protecting the cell-free sensor within a semipermeable compartment. The external addition of RNAse A, demonstrated here, serves as a proof-of-concept step towards cell-free detection in complex environments, such as biological samples or highly contaminated environmental samples.

TABLE 3
GFP expression in response to externally added NaF in
2:1 cholesterol:POPC vesicles with RNAse A in the surrounding
solution. Descriptive statistics of GFP expression in
populations of vesicles with RNAse present in the surrounding
solution in response to increasing concentrations of
externally added NaF. Statistical analysis was computed
compared to 0 mM NaF conditions.

| | | Mean | | Significantly | | |
|---|---|---|---|---|---|---|
| External NaF | | Fluorescence | | different than | Skew- | |
| concentration | | GFP/OA647 | SEM | 0 mM? | ness | Kurtosis |
| Extract only | | 2.152 | 0.013 | ns | 0.9806 | 2.661 |
| 0 | mM | 2.213 | 0.013 | | 0.9952 | 2.501 |
| 0.5 | mM | 3.071 | 0.026 | **** | 2.236 | 9.502 |
| 1 | mM | 3.289 | 0.022 | **** | 3.289 | 24.83 |
| 3 | mM | 2.930 | 0.023 | **** | 3.746 | 34.26 |
| 5 | mM | 2.662 | 0.018 | **** | 2.251 | 10.79 |

**** p £ 0.0001, nonsignificant (ns) p > 0.1234; p-values generated using a One-Way ANOVA and Tukey's Multiple Comparisons Test.

### Vesicle-Based Sensors can Incorporate a Colorimetric Readout and Detect Fluoride in Real-World Samples

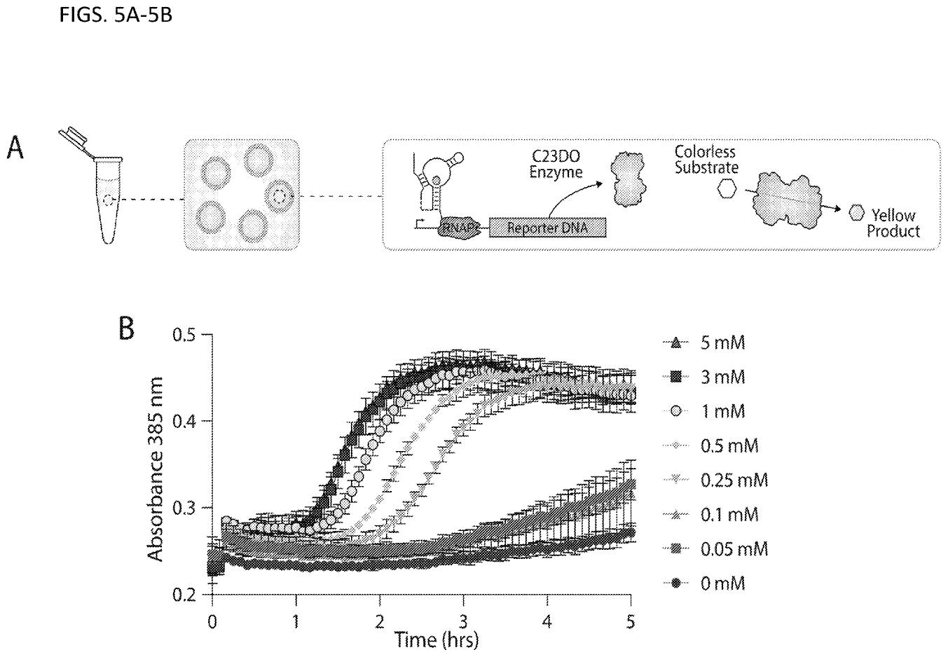

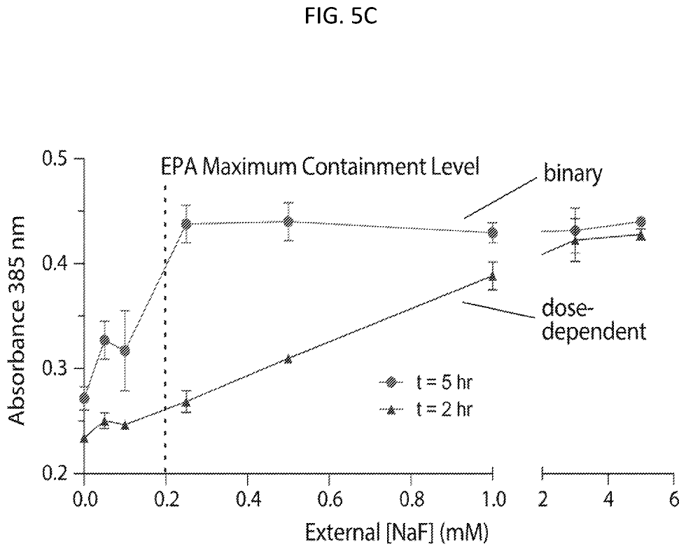

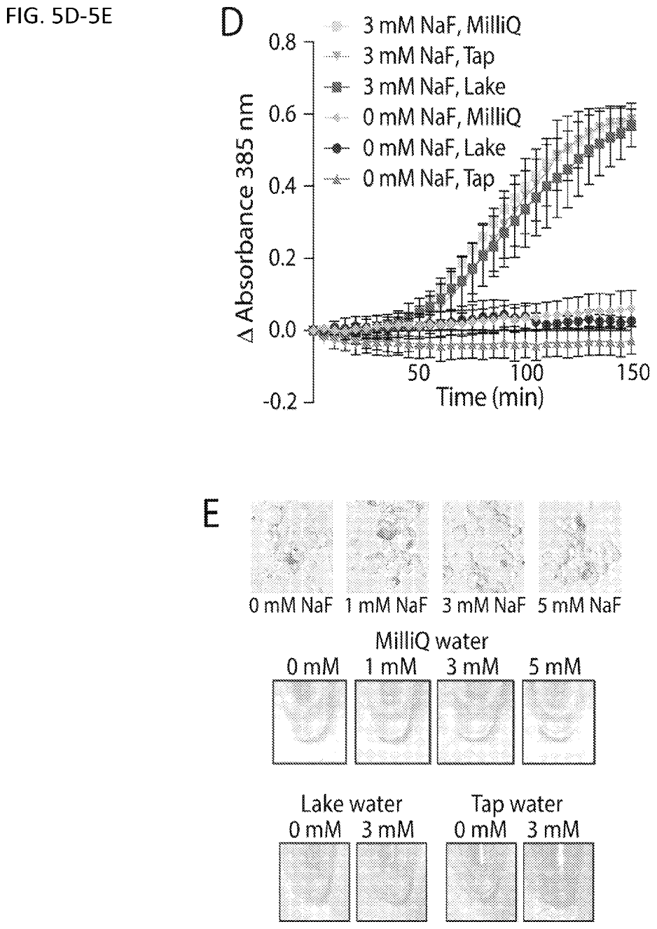

Finally, the inventors tested whether the results could be extended to conditions that would be more relevant for real-world environmental sensing. Although fluorescence is a common readout for many biological assays, GFP fluorescence inside vesicles is difficult to monitor with common equipment, particularly in non-laboratory settings. In addition, with the vesicle-based construct, GFP fluorescence was too low to measure in vesicle populations in bulk, necessitating more sensitive microscopy analysis methods. To address this limitation, the inventors coupled fluoride detection to an alternative reporter protein, catechol (2,3)-dioxygenase (C23DO) (3). In this system, the riboswitch “ON” state leads to the expression of C23DO, which catalyzes the conversion of its colorless substrate, catechol, to the yellow-colored 2-hydroxymuconate semialdehyde to generate a colorimetric response (FIG. 5A). In bulk conditions this construct exhibits a fast and robust response to fluoride, and the colorimetric output generated is clearly distinguishable by eye for both laboratory and field-collected water samples (3).

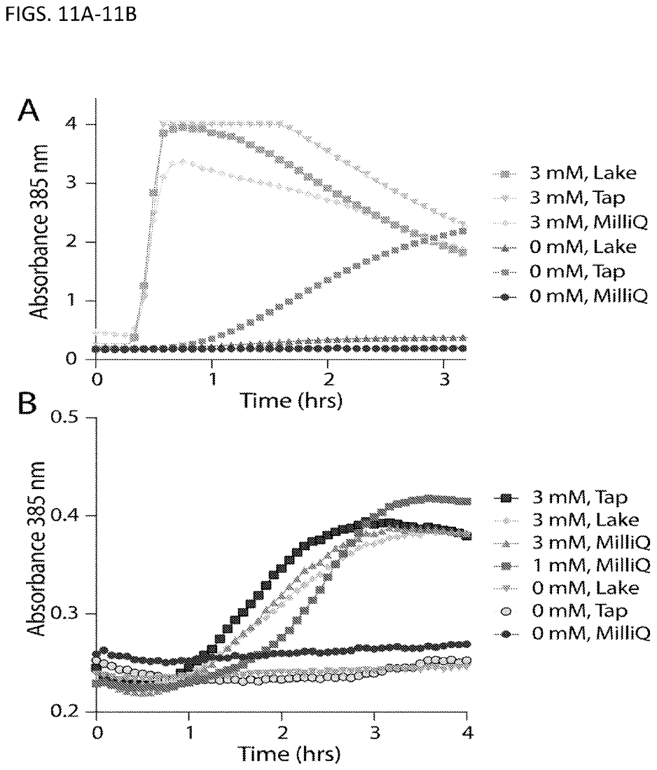

To investigate whether this enzymatic reporter could function within the sensor vesicles, the inventors encapsulated cell-free reactions with DNA coding for the riboswitch-C23DO construct and supplemented them with 1 mM catechol (FIG. 5A). The inventors then titrated NaF into the outer solution and monitored color changes in each population of vesicles via changes in absorbance at 385 nm. In contrast to the GFP-based readout, signal amplification from the enzyme-regulated construct allowed the inventors to assess absorbance changes in an entire population of vesicles rather than on a vesicle-by-vesicle basis. RNAse A was added to the outer vesicle solution to control for any unencapsulated reactions caused by vesicle lysis and to assess the membrane's ability to protect encapsulated sensor components. Sensor responses were significantly lower than those observed in bulk without RNAse (FIG. 11), however despite this reduction in signal magnitude, sensors exhibited significantly increased absorbance in response to increasing fluoride concentrations (FIG. 5B).

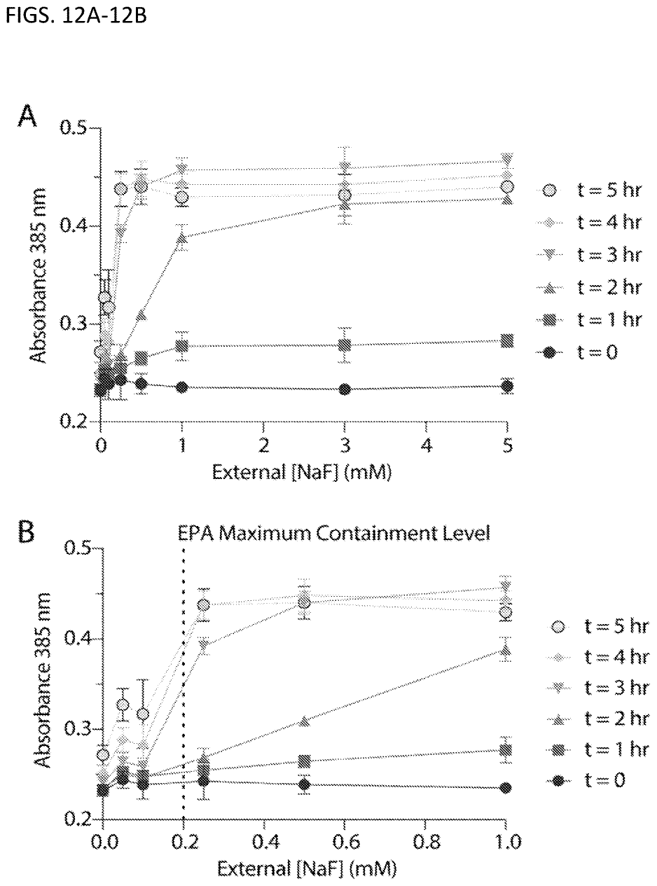

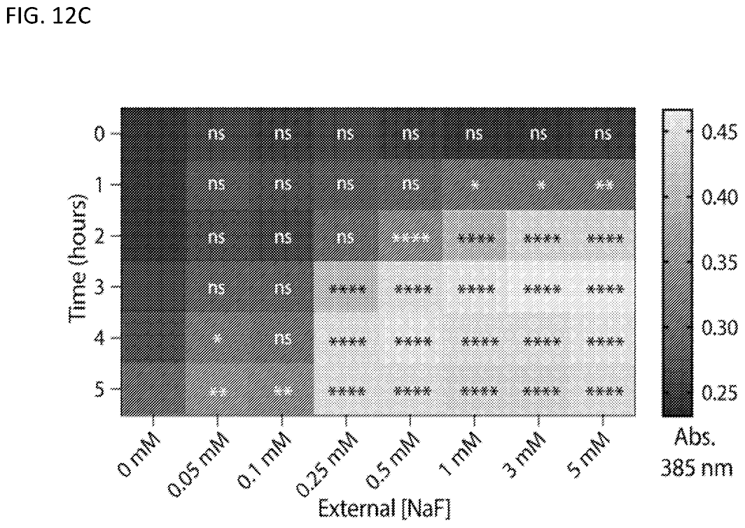

Vesicle sensors exhibited sensitivity to fluoride at a range of concentrations; here, readout time is an important consideration as enzymatic signal amplification can lower the resolution of a dose response curve and even reduce overall signal at long reaction times (FIG. 11) (3). Sensor responses to fluoride reach statistical significance at varying times depending on the external concentration of fluoride encountered (FIG. 12). This variability in response as a function of both time-to-detection and fluoride concentration can enable these sensors to serve as dose-responsive or binary (yes/no) sensors, depending on the time at which the response is assessed (FIGS. 5C and 12). Importantly, sensors could detect fluoride concentrations at levels consistent with the Environmental Protection Agency's maximum containment level for fluoride (~0.21 mM fluoride) and secondary containment standard (~0.1 mM) within 3 and 5 hours, respectively (FIGS. 5C and 12) (56). Responses were significant but noisy at 0.05 mM and 0.1 mM fluoride, however, indicating that these sensors would be most useful for detection of fluoride concentrations above the maximum containment limit of 0.2 mM (FIG. 5C). By accounting for both the detection time and the final absorbance of these colorimetric vesicle sensors, health-relevant concentrations of fluoride can ultimately be detected in either a binary or a dose-dependent manner.

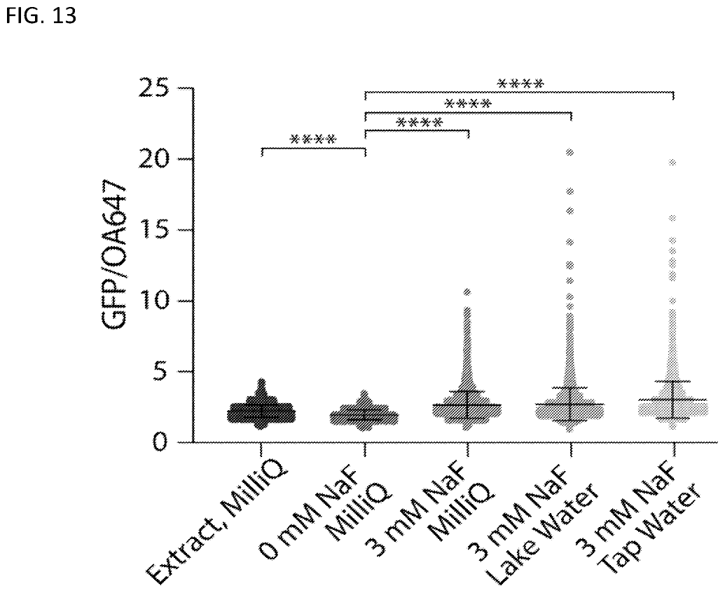

Finally, the inventors set out to test whether these sensors could be used to monitor fluoride concentrations in real-world samples. Water samples were collected from Lake Michigan and the Evanston, IL municipal tap water supply and each sample was used to prepare the vesicle outer solution supplemented with the small molecule components of the reaction. These outer solutions were supplemented with either 0 mM NaF or 3 mM NaF as well as RNAse A, which removes sensor activity from any ruptured vesicles, and vesicles were added to the sample. First assessed was the performance of encapsulated GFP sensors and an increased GFP expression in the presence of 3 mM NaF was observed, with slightly higher levels of GFP expression in both lake and tap water samples compared to those incubated with laboratory-grade Milli Q water (FIG. 13). The performance of encapsulated C23DO enzyme sensors was assessed next and similarly observed increased colorimetric outputs, measured via absorption at 385 nm, in all populations of enzyme-expressing vesicles incubated with 3 mM NaF (FIG. 5D). No increases in absorbance were observed in the absence of supplemented fluoride, with the exception of municipal tap water samples (FIG. 11). Color changes were visible by eye in tubes containing vesicles, and changes in color inside of individual vesicles could be observed on the microscope when imaged through the eyepiece (FIG. 5E). By incorporating a colorimetric detection method as demonstrated here, these sensors could ultimately be used to detect fluoride in samples containing inhospitable components, as well as in settings which prohibit the use of laboratory-based equipment. The results observed here highlight the feasibility of these vesicle-based sensors to detect environmentally relevant small molecules in complex, real-world samples, a step toward encapsulation to generate deployable cell-free sensors.

To the inventors' knowledge, this work represents the first demonstrated function of a transcriptionally regulated riboswitch encapsulated in bilayer vesicles. It was demonstrated that this encapsulated riboswitch can detect exogenous fluoride through permeability-based sensing, generating both fluorescent and colorimetric outputs. Additionally, it was shown that membrane composition can serve as an additional handle by which cell-free sensing reactions can be modulated by altering permeability and therefore the sensor's access to an analyte of interest. Further, it was demonstrated that encapsulation allows a cell-free sensor to outperform those in bulk solution when the solution contains degradative enzymes that inhibit sensor performance. Looking ahead toward sensor deployment, this work establishes that encapsulation within artificial cells can not only protect cell-free sensors from degradative sample components but can also allow analyte detection in real-world samples. Here, the encapsulation of a colorimetric reporter system allowed detection of fluoride in both a dose-responsive and binary manner at two EPA containment levels, even while in the presence of degradative sample components. While cell-free sensors have been previously used for the detection of environmental molecules of interest (1-9), encapsulation of these systems in artificial cells may ultimately diversify the contexts within which cell-free sensors can operate.

### Materials and Methods

### Chemicals

POPC (1-palmitoyl-2-oleoyl-glycero-3-phosphocholine) and cholesterol were purchased from Avanti Polar Lipids Inc. Oleic acid (OA), glycerol, sucrose, glucose, HPTS (8-Hydroxypyrene-1,3,6-trisulfonic acid trisodium salt), BioUltra Mineral Oil, phosphate-buffered saline (PBS), bovine serum albumin (BSA), and NaF were purchased from Millipore Sigma. 1.8k PEO-b-PBD polymer was purchased from Polymer Source. Ovalbumin-conjugated AlexaFluor 647 (OA647), calcein dye and HEPES buffer were purchased from Thermo Fisher. RNAse A was purchased from New England Biolabs.

### Plasmid Construction

Plasmids were assembled using Gibson assembly (New England Biolabs, Cat #E2611S) and purified using a Qiagen QIAfilter Midiprep Kit (QIAGEN, Cat #12143). Coding sequences of the plasmids consist of the crcB fluoride riboswitch from Bacillus cereus regulating either superfolder GFP (pJBL3752) or catechol 2,3-dioxygenase (pJBL7025), all expressed under constitutive Anderson promoter J23119. Plasmid sequences available on Addgene with accession numbers 128809 (pJBL3752) and 128810 (pJBL7025).

### Cell-Free Reaction Assembly

Cell-free extract and reactions were prepared according to established protocols (3, 39). Briefly, cell-free reactions were assembled by mixing cell extract, a reaction buffer containing the small molecules required for transcription and translation (NTPs, amino acids, buffering salts, crowding agents, and an energy source), and DNA templates and inducers at a ratio of approximately 30/30/40 (Table 4). Sucrose was added to a final concentration of 200 mM to allow vesicle formation through the density-dependent double emulsion technique. Each reaction was prepared on ice to 16.5 μL final volume in batches of 7. Reactions were prepared with 10 nM pJBL3752 (riboswitch-GFP plasmid) or pJBL7025 (riboswitch-enzyme plasmid)+1 mM catechol. Reaction master mix was assembled, then added to DNA, inducers, sucrose, and water to a final volume of 16.5 μL per reaction aliquot. For reactions containing volume marker, reaction mix was supplemented with 1.4 μL OA647. Preparation conditions were kept consistent between reactions, only varying NaF concentration or omitting DNA for extract-only controls.

TABLE 4
Components of cell-free protein expression reactions. All
components used to assemble cell-free expression reactions,
given as a fraction of the total reaction mixture.

| Component | Concentration | Units |
|---|---|---|
| Salt solution | 0.0667 | fraction |
| NTP Master mix | 0.0667 | fraction |
| Reagent mix | 0.0800 | fraction |
| Amino acids | 0.0400 | fraction |
| PEP | 0.0333 | fraction |
| Extract | 0.3000 | fraction |
| DNA/water/inducers | 0.4133 | fraction |

### Encapsulation of Cell-Free Reactions

Encapsulated sensors were prepared via water-in-oil double emulsion methods. Lipid films were prepared by mixing amphiphiles (lipid, cholesterol, fatty acid or polymers) in chloroform to a final amphiphile concentration of 25 mM at a volume of 200 μL. Films were dried onto the side of a glass vial under nitrogen gas, then placed in a vacuum oven overnight. 200 μL of BioUltra mineral oil was added to lipid films and heated at 80° C. for 30 minutes, followed by 10 seconds of vortexing to incorporate amphiphiles into the oil. Lipid/oil mixtures were cooled on to room temperature, then placed on ice during cell-free reaction assembly. Cell-free reactions were prepared on ice as described above. Reactions were layered on top of lipid/oil mixture, then vortexed for 30 seconds to form an emulsion. Emulsions were incubated at 4° C. for 5 minutes, then layered onto outer solution containing all small molecules required for transcription and translation, 100 mM HEPES buffer (pH 8), and 200 mM glucose, which is used to create a density gradient with the encapsulated sucrose. Samples were again incubated at 4° C. for 5 minutes, then centrifuged for 15 minutes at 18,000 rcf at 4° C. to spin the more dense, sucrose-containing vesicles into a pellet. Vesicle pellets were collected by pipette and placed into fresh Eppendorf tubes. Prepared vesicles were then added in 10 μL aliquots to 20 μL fresh outer solution supplemented with NaF, certain water samples and/or RNAse A (5 μg/mL final concentration). Osmolarity of NaF stock solution was adjusted to match that of the outer solution by adding glucose to minimize osmotic effects on vesicles.

### Cell-Free Protein Expression

For bulk assays, unencapsulated reactions were prepared as described above and added to 384-well plates. Protein expression was monitored at 37° C. in a SpectraMax i3x plate reader (Molecular Devices). GFP was monitored at ex: 485 nm, em: 510 nm. Catechol absorbance was monitored at 385 nm.

Encapsulated sensors with a colorimetric readout were monitored at an absorbance of 385 nm using a SpectraMax i3x plate reader at 37° C. until expression reached a plateau, about 2.5 hours, after which samples were removed from plates and placed into Eppendorf tubes or microscopy chambers for imaging. Images of tubes and through the microscope eyepiece were taken using an iPhone 8. Absorbance measurements in the plate reader are reported relative to initial absorbance to control for slight differences in vesicle concentration between vesicle preparations.

Encapsulated sensors expressing GFP were incubated in outer solution for 6 hours at 37° C., then imaged on a Nikon Ti2 inverted microscope. Imaging chambers were blocked with BSA for 20 minutes, then triple rinsed with 766 mOsm PBS. Vesicles were added to equiosmolar PBS and allowed to settle for 5 minutes before imaging. Images were taken using DIC, GFP (ex: 470, em: 525) and Cy 5.5 (ex: 650, em: 720) filters under 10× magnification, 20% laser intensity, and 1 second exposure. Images were analyzed using Nikon NIS-elements AR software Advanced Analysis tool (44): vesicles were selected using the OA647 channel. General analysis protocol was set with the following settings. Preprocessing: Local contrast, size 105, power 50%. Threshold minimum: 393. Smooth 1×, clean 1×. Size minimum: 2 μm. Return Mean GFP, Mean OA647, Max GFP.

### Encapsulated Dye Assays

HPTS assay: vesicles were prepared via thin film hydration with 33% Cholesterol and 66% POPC. Lipid and cholesterol in chloroform were dried onto the side of a glass vial under nitrogen gas to form a lipid film. Vesicle films were hydrated with HEPES+0.5 mM HPTS dye overnight at 60° C. Vesicles were extruded to 400 nm, purified via Size Exclusion Chromatography (SEC), and added to a 384-well plate with equiosmolar HEPES buffer+varying concentrations of NaCl and NaF. HPTS fluorescence was monitored with excitation at 405 and 450 nm and emission at 510 nm, as characterized by Hilburger et al. (59). HPTS fluorescence is reported as the ratio of emission intensities when excited at 450 nm/405 nm.

Calcein assay: vesicles were prepared via thin film hydration with 33% Cholesterol and 66% POPC. Lipid and cholesterol in chloroform were dried onto the side of a glass vial under nitrogen gas to form a lipid film. Vesicle films were hydrated with HEPES+20 mM calcein dye overnight at 60° C. Vesicles were extruded to 400 nm, purified via SEC, and added to a 384-well plate with equiosmolar HEPES buffer and increasing volumes of 0.02% glycerol solution or RNAse prepared in buffer to the same final glycerol concentration (1.25 μL corresponds to the concentration used for manuscript studies). Vesicles were incubated for 4 hours at 37° C. and calcein fluorescence was measured (ex: 495 nm, em: 515 nm). Vesicles were lysed with 1 μL 10% TritonX and total calcein fluorescence was measured to determine fraction release.

### Statistical Analysis

All graphing and statistical analysis was conducted in Graphpad Prism (60). Populations of vesicles were analyzed using One-Way ANOVA analysis with Tukey's Multiple Comparisons Test or Two-Way ANOVA and Dunnett's Multiple Comparisons Test and descriptive statistics. Significance is reported as follows: ns p>0.05, * p≤0.05, ** p≤0.01, *** p≤0.001, **** p<0.0001. Symbols and error bars on line plots represent mean and standard deviation, respectively.

### REFERENCES

1. P. Zhang, H. Feng, J. Yang, H. Jiang, H. Zhou, Y. Lu, Detection of inorganic ions and organic molecules with cell-free biosensing systems. Journal of Biotechnology. 300, 78-86 (2019).
2. A. Gräwe, A. Dreyer, T. Vornholt, U. Barteczko, L. Buchholz, G. Drews, U. L. Ho, M. E. Jackowski, M. Kracht, J. Lüders, T. Bleckwehl, L. Rositzka, M. Ruwe, M. Wittchen, P. Lutter, K. Müller, J. Kalinowski, A paper-based, cell-free biosensor system for the detection of heavy metals and date rape drugs. PLOS ONE. 14, e0210940 (2019).
3. W. Thavarajah, A. D. Silverman, M. S. Verosloff, N. Kelley-Loughnane, M. C. Jewett, J. B. Lucks, Point-of-Use Detection of Environmental Fluoride via a Cell-Free Riboswitch-Based Biosensor. ACS Synthetic Biology. 9, 10-18 (2020).
4. X. Liu, A. D. Silverman, K. K. Alam, E. Iverson, J. B. Lucks, M. C. Jewett, S. Raman, Design of a Transcriptional Biosensor for the Portable, On-Demand Detection of Cyanuric Acid. ACS Synthetic Biology. 9, 84-94 (2020).
5. A. D. Silverman, U. Akova, K. K. Alam, M. C. Jewett, J. B. Lucks, Design and Optimization of a Cell-Free Atrazine Biosensor. ACS Synthetic Biology. 9, 671-677 (2020).
6. P. L. Voyvodic, A. Pandi, M. Koch, I. Conejero, E. Valjent, P. Courtet, E. Renard, J.-L. Faulon, J. Bonnet, Plug-and-play metabolic transducers expand the chemical detection space of cell-free biosensors. Nature Communications. 10, 1697 (2019).
7. T. T. M. Duyen, H. Matsuura, K. Ujiie, M. Muraoka, K. Harada, K. Hirata, Paper-based colorimetric biosensor for antibiotics inhibiting bacterial protein synthesis. Journal of Bioscience and Bioengineering. 123, 96-100 (2017).
8. T. Pellinen, T. Huovinen, M. Karp, A cell-free biosensor for the detection of transcriptional inducers using firefly luciferase as a reporter. Analytical Biochemistry. 330, 52-7 (2004).
9. S. Gupta, S. Sarkar, A. Katranidis, J. Bhattacharya, Development of a Cell-Free Optical Biosensor for Detection of a Broad Range of Mercury Contaminants in Water: A Plasmid DNA-Based Approach. ACS Omega. 4, 9480-9487 (2019).
10. K. Pardee, A. A. Green, M. K. Takahashi, D. Braff, G. Lambert, J. W. Lee, T. Ferrante, D. Ma, N. Donghia, M. Fan, N. M. Daringer, I. Bosch, D. M. Dudley, D. H. O'Connor, L. Gehrke, J. J. Collins, Rapid, Low-Cost Detection of Zika Virus Using Programmable Biomolecular Components. Cell. 165, 1255-1266 (2016).
11. D. Ma, L. Shen, K. Wu, C. W. Diehnelt, A. A. Green, Low-cost detection of norovirus using paper-based cell-free systems and synbody-based viral enrichment. Synthetic Biology. 3, ysy018 (2018).
12. M. Verosloff, J. Chappell, K. L. Perry, J. R. Thompson, J. B. Lucks, PLANT-Dx: A Molecular Diagnostic for Point-of-Use Detection of Plant Pathogens. ACS Synthetic Biology. 8, 902-905 (2019).
13. M. K. Takahashi, X. Tan, A. J. Dy, D. Braff, R. T. Akana, Y. Furuta, N. Donghia, A. Ananthakrishnan, J. J. Collins, A low-cost paper-based synthetic biology platform for analyzing gut microbiota and host biomarkers. Nature Communications. 9, 3347 (2018).
14. K. Y. Wen, L. Cameron, J. Chappell, K. Jensen, D. J. Bell, R. Kelwick, M. Kopniczky, J. C. Davies, A. Filloux, P. S. Freemont, A Cell-Free Biosensor for Detecting Quorum Sensing Molecules in P. aeruginosa-Infected Respiratory Samples. ACS Synthetic Biology. 6, 2293-2301 (2017).
15. J. P. Hunt, R. J. Barnett, H. Robinson, M. Soltani, J. A. D. Nelson, B. C. Bundy, Rapid sensing of clinically relevant glutamine concentrations in human serum with metabolically engineered E. coli-based cell-free protein synthesis. Journal of Biotechnology. 325, 389-394 (2021).
16. J. P. Hunt, E. L. Zhao, T. J. Free, M. Soltani, C. A. Warr, A. B. Benedict, M. K. Takahashi, J. S. Griffitts, W. G. Pitt, B. C. Bundy, Towards detection of SARS-COV-2 RNA in human saliva: A paper-based cell-free toehold switch biosensor with a visual bioluminescent output. New Biotechnology. 66, 53-60 (2022).
17. A. S. M. Salehi, M. J. Shakalli Tang, M. T. Smith, J. M. Hunt, R. A. Law, D. W. Wood, B. C. Bundy, Cell-Free Protein Synthesis Approach to Biosensing hTRβ-Specific Endocrine Disruptors. Analytical Chemistry. 89, 3395-3401 (2017).
18. M. A. Boyd, N. P. Kamat, Designing Artificial Cells towards a New Generation of Biosensors. Trends in Biotechnology. 39, 927-939 (2021).
19. M. Soltani, J. P. Hunt, B. C. Bundy, Rapid RNase inhibitor production to enable low-cost, on-demand cell-free protein synthesis biosensor use in human body fluids. Biotechnology and Bioengineering. 118, 3973-3983 (2021).
20. M. P. McNerney, Y. Zhang, P. Steppe, A. D. Silverman, M. C. Jewett, M. P. Styczynski, Point-of-care biomarker quantification enabled by sample-specific calibration. Science Advances. 5, eaax4473 (2019).
21. K. P. Adamala, D. A. Martin-Alarcon, K. R. Guthrie-Honea, E. S. Boyden, Engineering genetic circuit interactions within and between synthetic minimal cells. Nature Chemistry. 9, 431-439 (2017).
22. C. Tan, S. Saurabh, M. P. Bruchez, R. Schwartz, P. LeDuc, Molecular crowding shapes gene expression in synthetic cellular nanosystems. Nature Nanotechnology. 8, 602-608 (2013).
23. Z. Nourian, C. Danelon, Linking genotype and phenotype in protein synthesizing liposomes with external supply of resources. ACS Synthetic Biology. 2, 186-93 (2013).
24. T. P. de Souza, P. Stano, P. L. Luisi, The Minimal Size of Liposome-Based Model Cells Brings about a Remarkably Enhanced Entrapment and Protein Synthesis. ChemBioChem. 10, 1056-1063 (2009).
25. R. Sakamoto, V. Noireaux, Y. T. Maeda, Anomalous Scaling of Gene Expression in Confined Cell-Free Reactions. Scientific Reports. 8, 7364 (2018).
26. A. D. Silverman, A. S. Karim, M. C. Jewett, Cell-free gene expression: an expanded repertoire of applications. Nature reviews. Genetics. 21, 151-170 (2020).
27. S. W. Schaffter, R. Schulman, Building in vitro transcriptional regulatory networks by 27. successively integrating multiple functional circuit modules. Nature Chemistry. 11, 829-838 (2019).
28. R. Lentini, S. P. Santero, F. Chizzolini, D. Cecchi, J. Fontana, M. Marchioretto, C. del Bianco, J. L. Terrell, A. C. Spencer, L. Martini, M. Forlin, M. Assfalg, M. D. Serra, W. E. Bentley, S. S. Mansy, Integrating artificial with natural cells to translate chemical messages that direct E. coli behaviour. Nature Communications. 5, 4012 (2014).
29. L. Martini, S. S. Mansy, Cell-like systems with riboswitch controlled gene expression. Chemical Communications. 47, 10734-10736 (2011).
30. M. Dwidar, Y. Seike, S. Kobori, C. Whitaker, T. Matsuura, Y. Yokobayashi, Programmable Artificial Cells Using Histamine-Responsive Synthetic Riboswitch. Journal of the American Chemical Society. 141, 11103-11114 (2019).
31. T. Janas, T. Janas, M. Yarus, Specific RNA binding to ordered phospholipid bilayers. Nucleic Acids Research. 34, 2128-36 (2006).
32. T. Czerniak, J. P. Saenz, Lipid membranes modulate the activity of RNA through sequence-dependent interactions. Proceedings of the National Academy of Sciences. 119, 2022 (2022).
33. E. J. Strobel, L. Cheng, K. E. Berman, P. D. Carlson, J. B. Lucks, A ligand gated strand displacement mechanism for ZTP riboswitch transcription control. Nature Chemical Biology. 15, 1067 (2019).
34. UNICEF, WHO, “Progress on Household Drinking Water, Sanitation and Hygiene 2000-2017: Special focus on inequalities” (2019), (available at https://www.unicef.org/reports/progress-on-drinking-water-sanitation-and-hygiene-2019).
35. R. Damania, S. Desbureaux, A.-S. Rodella, J. Russ, E. Zaveri, Quality Unknown: The Invisible Water Crisis (Washington, DC: World Bank, 2019; http://hdl.handle.net/10986/32245).
36. R. Fuge, Fluorine in the environment, a review of its sources and geochemistry. Applied Geochemistry. 100, 393-406 (2019).
37. P. B. McMahon, C. J. Brown, T. D. Johnson, K. Belitz, B. D. Lindsey, Fluoride occurrence in United States groundwater. The Science of the Total Environment. 732, 139217 (2020).
38. K. E. Watters, E. J. Strobel, A. M. Yu, J. T. Lis, J. B. Lucks, Cotranscriptional folding of a riboswitch at nucleotide resolution. Nature Structural & Molecular Biology. 23, 1124-1131 (2016).
39. A. D. Silverman, N. Kelley-Loughnane, J. B. Lucks, M. C. Jewett, Deconstructing Cell-Free Extract Preparation for in Vitro Activation of Transcriptional Genetic Circuitry. ACS Synthetic Biology. 8, 403-414 (2019).
40. O. Barbier, L. Arreola-Mendoza, L. M. del Razo, Molecular mechanisms of fluoride toxicity. Chemico-Biological Interactions. 188, 319-33 (2010).
41. S. Pautot, B. J. Frisken, D. A. Weitz, Production of Unilamellar Vesicles Using an Inverted Emulsion. Langmuir. 19, 2870-2879 (2003).
42. D. T. Gonzales, N. Yandrapalli, T. Robinson, C. Zechner, T. Y. D. Tang, Cell-Free Gene Expression Dynamics in Synthetic Cell Populations. ACS Synthetic Biology. 11, 205-215 (2022).
43. K. Nishimura, T. Matsuura, K. Nishimura, T. Sunami, H. Suzuki, T. Yomo, Cell-free protein synthesis inside giant unilamellar vesicles analyzed by flow cytometry. Langmuir. 28, 8426-8432 (2012).
44. Nikon Instruments Inc., NIS-Elements Advanced Research, (available at https://www.microscope.healthcare.nikon.com/products/software/nis-elements/nis-elements-advanced-research).
45. K. Nishimura, S. Tsuru, H. Suzuki, T. Yomo, Stochasticity in Gene Expression in a Cell-Sized Compartment. ACS Synthetic Biology. 4, 566-576 (2014).
46. Z. Nourian, W. Roelofsen, C. Danelon, Triggered Gene Expression in Fed-Vesicle Microreactors with a Multifunctional Membrane. Angewandte Chemie International Edition. 51, 3114-3118 (2012).
47. H. Saito, Y. Kato, M. le Berre, A. Yamada, T. Inoue, K. Yosikawa, D. Baigl, Time-Resolved Tracking of a Minimum Gene Expression System Reconstituted in Giant Liposomes. ChemBioChem. 10, 1640-1643 (2009).
48. D. Garenne, V. Noireaux, Analysis of Cytoplasmic and Membrane Molecular Crowding in Genetically Programmed Synthetic Cells. Biomacromolecules. 21, 2808-2817 (2020).
49. J. Gutknecht, A. Walter, Hydrofluoric and nitric acid transport through lipid bilayer membranes. Biochimica et Biophysica Acta. 644, 153-6 (1981).
50. D. Papahadjopoulos, S. Nir, S. Ohki, Permeability properties of phospholipid membranes: Effect of cholesterol and temperature. Biochimica et Biophysica Acta (BBA)-Biomembranes. 266, 561-583 (1972).
51. B. M. Discher, Y.-Y. Won, D. S. Ege, J. C.-M. Lee, F. S. Bates, D. E. Discher, D. A. Hammer, Polymersomes: Tough Vesicles Made from Diblock Copolymers. Science. 284, 1143-1146 (1999).
52. M. L. Jacobs, M. A. Boyd, N. P. Kamat, Diblock copolymers enhance folding of a mechanosensitive membrane protein during cell-free expression. Proceedings of the National Academy of Sciences. 116, 4031-4036 (2019).
53. H. Jespersen, J. H. Andersen, H. J. Ditzel, O. G. Mouritsen, Lipids, curvature stress, and the action of lipid prodrugs: free fatty acids and lysolipid enhancement of drug transport across liposomal membranes. Biochimie. 94, 2-10 (2012).
54. P.-A. Monnard, D. W. Deamer, Membrane self-assembly processes: steps toward the first cellular life. The Anatomical Record. 268, 196-207 (2002).
55. Jiang, N., Ding, X., Lu, Y., Development of a robust Escherichia coli-based cell-free protein synthesis application platform. Biochem Eng. J. 165, 107830 (2021).
56. Environmental Protection Agency (EPA), “National primary drinking water regulations” (2009), (available at https://www.epa.gov/ground-water-and-drinking-water/national-primary-drinking-water-regulations).
57. N. S. Kruyer, W. Sugianto, B. I. Tickman, D. Alba Burbano, V. Noireaux, J. M. Carothers, P. Peralta-Yahya, Membrane Augmented Cell-Free Systems: A New Frontier in Biotechnology. ACS Synthetic Biology. 10, 670-681 (2021).
58. J. T. Lazar, J. J. Tabor, Bacterial two-component systems as sensors for synthetic biology applications. Current Opinion in Systems Biology. 28, 100398 (2021).
59. C. E. Hilburger, M. L. Jacobs, K. R. Lewis, J. A. Peruzzi, N. P. Kamat, Controlling Secretion in Artificial Cells with a Membrane AND Gate. ACS Synthetic Biology. 8, 1224-1230 (2019).

1. A composition comprising:
a cell-free system; and
a construct encoding a riboswitch coupled to a reporter protein;
wherein the riboswitch is responsive to an analyte; and
wherein the composition is encapsulated within a membrane.

2. The composition of claim 1, wherein the membrane is a bilayer membrane.

3. The composition of claim 1 or 2, wherein the membrane comprises 1-palmitoyl-2-oleoyl-glycero-3-phosphocholine (POPC).

4. The composition of claim 3, wherein the membrane further comprises at least one of cholesterol, oleic acid, and poly(ethylene oxide)-b-poly(butadiene) (PEO-b-PBD) diblock copolymer.

5. The composition of claim 3, wherein the membrane comprises a 2:1 cholesterol:POPC lipid composition.

6. The composition of any one of claims 1-5, wherein the analyte is fluoride.

7. The composition of claim 6, wherein the riboswitch is a Bacillus cereus riboswitch.

8. The composition of any one of claims 1-7, wherein the reporter protein is a fluorescent reporter or a colorimetric reporter.

9. The composition of claim 8, wherein the reporter protein is a fluorescent reporter, and wherein the composition further comprises a volume marker.

10. The composition of any one of claims 1-9, wherein the reporter protein is green fluorescent protein (GFP).

11. The composition of claim 9, wherein the reporter protein is GFP, and wherein the volume marker comprises OA647.

12. The composition of any one of claims 1-8, wherein the reporter protein is catechol (2,3)-dioxygenase (C23DO), and wherein the composition further comprises catechol.

13. The composition of claim 12, wherein the composition comprises about 1 mM catechol.

14. A method of detecting an analyte in a sample, the method comprising:
contacting the sample with a composition comprising a cell-free system and a construct encoding a riboswitch coupled to a reporter protein, wherein the composition is encapsulated within a membrane; and
detecting a signal emitted by the reporter protein;
wherein the riboswitch is responsive to the analyte; and
wherein if the analyte is present in the sample, the signal is emitted.

15. The method of claim 14, wherein the membrane is a bilayer membrane.

16. The method of claim 14 or 15, wherein the membrane comprises 1-palmitoyl-2-oleoyl-glycero-3-phosphocholine (POPC).

17. The method of claim 16, wherein the membrane further comprises at least one of cholesterol, oleic acid, and poly(ethylene oxide)-b-poly(butadiene) (PEO-b-PBD) diblock copolymer.

18. The method of claim 17, wherein the membrane comprises a 2:1 cholesterol:POPC lipid composition.

19. The method of any one of claims 14-18, wherein the analyte is fluoride, and wherein the riboswitch is a fluoride-responsive riboswitch.

20. The method of claim 19, wherein the fluoride-responsive riboswitch is a Bacillus cereus riboswitch.

21. The method of any one of claims 14-20, wherein the reporter protein is a fluorescent reporter or a colorimetric reporter.

22. The method of claim 21, wherein the reporter protein is a fluorescent reporter, and wherein the composition further comprises a volume marker.

23. The method of any one of claims 14-21, wherein the reporter protein is green fluorescent protein (GFP).

24. The method of claim 22, wherein the reporter protein is GFP; and wherein the volume marker is OA647.

25. The method of any one of claims 14-21, wherein the reporter protein is catechol (2,3)-dioxygenase (C23DO), and wherein the composition further comprises catechol.

26. The method of claim 25, wherein the composition comprises about 1 mM catechol.

27. The method of any one of claims 14-26, further comprising incubating the sample in the system for at least 90 minutes before detecting the signal.

28. The method of any one of claims 14-26, further comprising incubating the sample in the system for at least three hours before detecting the signal.

29. The method of any one of claims 14-26, further comprising incubating the sample in the system for between about three and about five hours before detecting the signal.

30. The method of any one of claims 14-29, further comprising adding an RNAse to the sample before contacting the sample with the composition.

31. The method of any one of claims 14-30, wherein the sample is a water sample.

32. A kit comprising the composition of any one of claims 1-13, wherein the composition is in a container that a sample can be added to.

33. The kit of claim 32, wherein the container is a clear container, and wherein the reporter protein is a colorimetric reporter that emits a signal visible to the eye.
